(19) United States
(12) Patent Application Publication
(10) Pub. No.: US 2008/0153135 A1
(43) Pub. Date: Jun. 26, 2008

(54) Methods and apparatus for conducting amplification reactions on high density hydrophilic patterned microplates

(76) Inventor: Timothy Z. Liu, Fremont, CA (US)

Correspondence Address: HARNESS, DICKEY & PIERCE, P.L.C., P.O. BOX 828, BLOOMFIELD HILLS, MI, 48303, US

(21) Appl. No.: 11/642,764

(22) Filed: Dec. 20, 2006

(51) Int. Cl. C12P 19/38; C12M 3/00

(52) U.S. Cl. 435/91.2; 435/288.4

(57) ABSTRACT

A microplate for use in performing PCR on a target. The microplate having a substrate with a hydrophobic surface and a plurality of hydrophilic reaction spots on the hydrophobic surface of the substrate. Each of the reaction spots having a capacity to retain less than 5 nanoliters of an aqueous solution. Each of the plurality of hydrophilic reaction spots having a primer and a detection probe anchored.

### INTRODUCTION

Currently, genomic analysis, including that of the estimated 30,000 human genes, is a major focus of basic and applied biochemical and pharmaceutical research. Such analysis can aid in developing diagnostics, medicines, and therapies for a wide variety of disorders. However, the complexity of the human genome and the interrelated functions of genes often make this task difficult. There is a continuing need for methods and apparatus to aid in such analysis.

### DRAWINGS

The skilled artisan will understand that the drawings, described herein, are for illustration purposes only. The drawings are not intended to limit the scope of the present teachings in any way.

FIG. 1 is a top perspective view illustrating a plurality of reaction spots on a hydrophobic substrate in accordance with some embodiments;

FIG. 2. is an enlarged perspective view illustrating a plurality of reaction spots on a hydrophobic substrate in accordance with some embodiments;

FIG. 3 is a cross-sectional view illustrating a solution comprising polyvinylalcohol on a hydrophobic substrate in accordance with some embodiments;

FIG. 4 is a cross-sectional view illustrating at least one polynucleotide conjugated to a reaction spot using a cross-linker in accordance with some embodiments;

FIG. 5 is a cross-sectional view illustrating at least one polynucleotide anchored to a reaction spot employing a cleavable site in accordance with some embodiments;

FIG. 6 is a cross-sectional view of at least one biotinylated polynucleotide complex bound to an agrose fiber that is part of a reaction spot in accordance with some embodiments;

FIG. 7 is a cross-sectional view of at least one polynucleotide bound to at least one reaction spot employing streptavidin and comprising a cleavable site in accordance with some embodiments;

FIG. 8 is a cross-sectional view illustrating at least one polynucleotide bound to a dimethyl acrylamide monomer employing a cleavable site in accordance with some embodiments;

FIG. 9 is a cross-sectional schematic view illustrating an apparatus for measuring a change in at least one of the plurality of reaction chambers in accordance with some embodiments;

FIG. 10 is a cross-sectional view illustrating a plurality of reaction chambers on a hydrophobic substrate in accordance with some embodiments;

FIG. 11 is a perspective view illustrating a microplate comprising a plurality of reaction chambers, a seal, and a cover in accordance with some embodiments;

FIGS. 12(a)-(b) are images from a microscope illustrating amphiphilic micelles comprising at least a polystyrene portion and a polynucleotide in accordance with some embodiments;

FIGS. 13(a)-(h) are images from a microscope illustrating a reaction spot comprising a polystyrene-polynucleotide complex after hybridization in accordance with some embodiments; and

FIGS. 14(a)-(c) are images from a microscope illustrating 1 nl reaction spots comprising a polystyrene-polynucleotide complex after hybridization in accordance with some embodiments.

### DESCRIPTION OF VARIOUS EMBODIMENTS

The following description of some embodiments is merely exemplary in nature and is in no way intended to limit the present teachings, applications, or uses. Although the present teachings will be discussed in some embodiments as relating to polynucleotide amplification, such as PCR, such discussion should not be regarded as limiting the present teaching to only such applications.

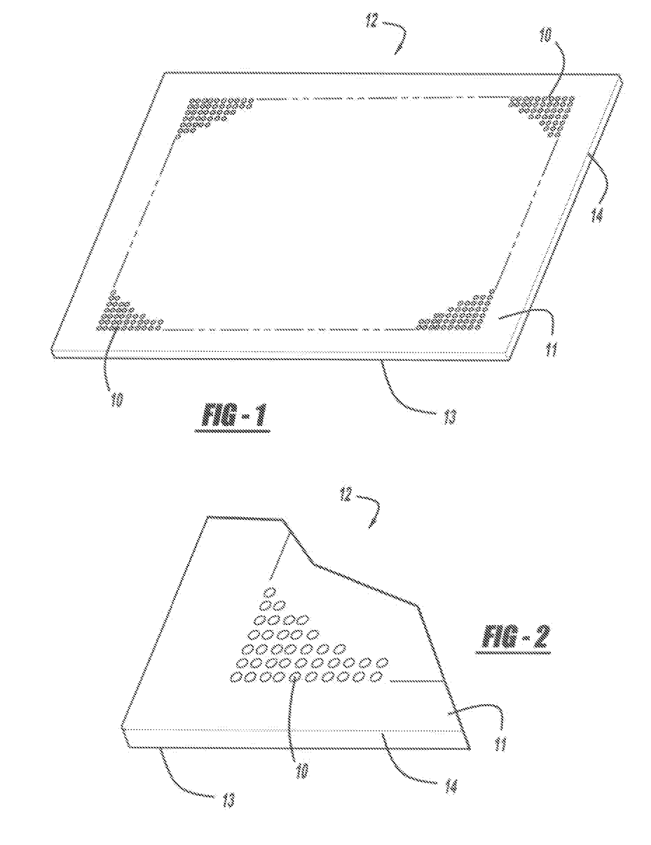

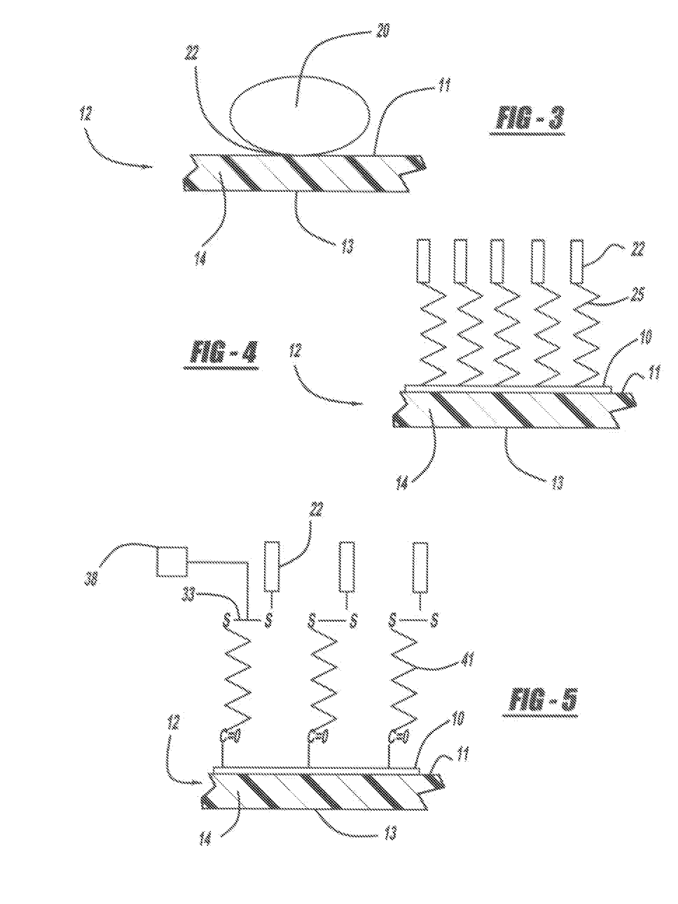

Referring to FIGS. 1 and 2, in some embodiments, a microplate 12 is provided comprising a substrate 14 for use, in part, in the performance of an analytical method or chemical reaction. In some embodiments, microplate 12 can comprise a plurality of reaction spots or material retention regions 10 configured to hold or support a material such as, for example, an assay 1000.

In some embodiments, assay 1000 can comprise any material that is useful in, the subject of, a precursor to, or a product of an analytical method or chemical reaction. In some embodiments for amplification and/or detection of polynucleotides, assay 1000 comprises one or more reagents (such as PCR master mix, as described further herein); an analyte (such as a biological sample comprising DNA, a DNA fragment, cDNA, RNA, or any other nucleic acid sequence); one or more primers; one or more primer sets; one or more detection probes; components thereof; and combinations thereof. In some embodiments, assay 1000 comprises a homogenous solution of a DNA sample, at least one primer set, at least one detection probe, a polymerase, and a buffer, as used in a homogenous assay (described further herein). In some embodiments, assay 1000 can comprise an aqueous solution of at least one analyte, at least one primer set, at least one detection probe, and a polymerase. In some embodiments, assay 1000 can be an aqueous homogenous solution. In some embodiments, assay 1000 can comprise at least one of a plurality of different detection probes and/or primer sets to perform multiplex PCR, which can be useful, for example, when analyzing a whole genome (e.g., 20,000 to 30,000 genes, or more) or other large numbers of genes or sets of genes.

Still referring to FIGS. 1 and 2, in some embodiments, substrate 14 can comprise a substantially planar first surface 11 and an opposing second surface 13. In some embodiments, microplate 12 and/or substrate 14 thereof can have dimensions such that microplate 12 can be used in conventional PCR equipment. In some embodiments, microplate 12 can be from about 50 to about 200 mm in width, or from about 50 to about 200 mm in length. In some embodiments, microplate 12 can be from about 50 to about 100 mm in width, or from about 100 to about 150 mm in length. In some embodiments, microplate 12 can be about 72 mm wide and about 108 mm in length. In order to facilitate use with existing equipment, robotic implementations and instrumentations, in some embodiments, microplate 12 can conform to standards specified by the American National Standards Institute (ANSI) and the Society of Biomolecular Screening (SBS), published January 2004 (ANSI/SBS 3-2004). In some embodiments, the footprint dimensions of microplate 12 can be about 127.76 mm (5.0299 inches) in length and about 85.48 mm (3.3654 inches) in width.

First surface 11 can be configured to include at least some of the plurality of reaction spots 10 therein or thereon. In some embodiments, such plurality of reaction spots 10 can be hydrophilic spots or pads, and the like.

In some embodiments, microplate 12 can be used for single-use, wherein it can be filled or otherwise used with a single assay for a single experiment or set of experiments, and can be thereafter discarded. In some embodiments, microplate 12 can be used for multiple-use, wherein it can be operable for use in a plurality of experiments or sets of experiments. In some embodiments, microplate 12 can be used in amplifying polynucleotides in a liquid sample comprising a plurality of polynucleotide targets.

In some embodiments, substrate 14 can be made of any material which is suitable for conducting amplification of polynucleotides such as, for example, by PCR. In some embodiments, the material can be substantially non-reactive with polynucleotide targets, primers and reagents employed in amplification reactions. In some embodiments, the material can be substantially non-reactive with assay 1000. In some embodiments, the material does not interfere with detecting a signal from an amplification reaction. In some embodiments in which imaging can be performed by detection of fluorescent labeled reagents, the material can be opaque to transmission of light emitted by the fluorescent labeled reagents such as, for example, a detection probe. In some embodiments, substrate 14 can comprise glass, plastic, silicon, quartz, nylon, metal, borosilicate, fused silica, polytetrafluoroethylene, polyethylene, polypropylene, polycarbonate, polyolefin, polyetherketone, polyamideimide, polydimethyl siloxane, polystyrene, or combinations thereof. In some embodiments, substrate 14 can be glass, such as, for example, borosilicate, flint glass, crown glass, float glass, or fused silica. In some embodiments, substrate 14 can be a high temperature plastic, such as, for example, polycarbonate, polyolefin, polytetrafluoroethylene, polyetherketone, polyamideimide, polypropylene, polydimethyl siloxane, and combinations thereof. In some embodiments, a polynucleotide can be a polymeric chain of nucleotides of any length. In some embodiments, a polynucleotide can include, but not limited to, DNA cDNA, RNA, DNA fragments, RNA fragments, oligonucleotides, PCR primers, detection probes, hybridization sites, targets, ligation sites, probes, nucleic acid sequences, or the like. In some embodiments, a polynucleotide can be from a natural source, such as, for example a plant, a bacteria, an animal, or a human, or can be synthetically derived. In some embodiments, a polynucleotide can be derived from any organism or other source including, but not limited to, prokaryotes, eukaryotes, plants, animals, and viruses, as well as synthetic nucleic acids, for example. In some embodiments, polynucleotides can originate from any of a wide variety of sample types, such as cell nuclei (such as, for example, genomic DNA), whole cells, tissue samples, phage, plasmids, mitrochondria, and the like. In some embodiments, polynucleotides can contain DNA, RNA, and/or variants or modifications thereof.

In some embodiments, at least one of the plurality of reaction spots 10 can be a defined area on substrate 14 which localizes reagents employed in the amplification of at least one polynucleotide target in sufficient quantity, proximity, and isolation from adjacent areas on substrate 14 (such as other of the plurality of reaction spots 10 on substrate 14), so as to facilitate amplification of one or more polynucleotide targets in the at least one of the plurality of reaction spots 10. In some embodiments, localization can be accomplished by physical and chemical modalities, including physical containment of reagents in one dimension and chemical containment in one or more other dimensions. In some embodiments, physical containment can be effected by first surface 11 of substrate 14 itself, such that first surface 11 forms the bottom of at least one of the plurality of reaction spots 10. In some embodiments, containment of the at least one of the plurality of reaction spots 10 in other dimensions can be effected primarily through chemical modalities, such as through the chemical characteristics of first surface 11 of substrate 14 surrounding the at least one of the plurality of reaction spots 10, containment fluids, binding of one or more reagents to first surface 11, and combinations thereof.

In some embodiments, the at least one of the plurality of reaction spots 10 comprises an amplification reagent, wherein the amplification reagent can be affixed or otherwise contained on or in the at least one of the plurality of reaction spots 10 in such a manner so as to be available for an amplification reaction method of these teachings. In some embodiments, the amplification reagent can be a reagent which can be used in an amplification reaction such as, for example, PCR. In some embodiments, assay 1000 comprises an amplification reagent. In some embodiments, the amplification reagent comprises at least one primer. In some embodiments, the amplification reagent comprises at least one primer pair.

In some embodiments, the at least one of the plurality of reaction spots 10 comprises a detection probe comprising a reagent, which can be affixed or otherwise contained on or in the at least one of the plurality of reaction spots 10 in such a manner so as to be available for hybridization to a polynucleotide target of interest. In some embodiments, assay 1000 comprises a detection probe. In some embodiments, the at least one of the plurality of reaction spots 10 comprises a primer pair for a specific polynucleotide target, and a detection probe for that polynucleotide target.

In some embodiments, material retention regions of microplate 12 can comprise a plurality of reaction spots 10 on first surface 11 of the microplate 12. In some embodiments, at least one of the plurality of reaction spots 10 can be an area on substrate 14 which localizes, at least in part by non-physical means, assay 1000. In some embodiments, assay 1000 can be localized in sufficient quantity, and isolation from adjacent areas on microplate 12, so as to facilitate an analytical method or chemical reaction (such as, for example, amplification of one or more polynucleotide targets) in a material retention region. Such localization can be accomplished by physical and chemical modalities, including, for example, physical containment of reagents in one dimension and chemical containment in one or more other dimensions, as discussed above.

In some embodiments, first surface 11 of the microplate 12 comprises an enhanced surface which can comprise a physical or chemical modality on or in first surface 11 of microplate 12 so as to enhance support of, or filling of, assay 1000 in a material retention region such as at least one of a plurality of reaction spots 10. Such modifications can include chemical treatment of first surface 11, or coating first surface 11. In some embodiments, such chemical treatment can comprise chemical treatment or modification of first surface 11 of microplate 12 so as to form relatively hydrophilic and hydrophobic areas. In some embodiments, a surface tension array can be formed comprising a pattern of hydrophilic sites forming a plurality of reaction spots 10 on a hydrophobic substrate, such that the hydrophilic sites can be spatially segregated by hydrophobic regions. Reagents delivered to the surface tension array can be constrained by surface tension difference between hydrophilic and hydrophobic areas.

In some embodiments, hydrophobic sites can be formed on first surface 11 of substrate 14 by forming first surface 11, or chemically treating it, with compounds comprising alkyl groups. In some embodiments, hydrophilic sites can be formed on first surface 11 of substrate 14 by forming the surface, or chemically treating it, with compounds comprising free amino, hydroxyl, carboxyl, thiol, amido, halo, or sulfate groups. In some embodiments, the free amino, hydroxyl, carboxyl, thiol, amido, halo, or sulfate group of the hydrophilic sites can be covalently coupled with a linker moiety (such as, for example, polylysine, hexethylene glycol, and polyethylene glycol). A variety of methods of forming surface tension arrays useful herein can be found in the art and examples of such methods can be found in U.S. Pat. Nos. 5,474,796 and 5,985,551.

In some embodiments, a surface tension array can be formed by photoresist methods. In some embodiments, a surface tension array can be formed by coating substrate 14 with a photoresist substance and then using a generic photomask to define array patterns on substrate 14 by exposing the array patterns to light. The exposed surface can be reacted with a suitable reagent to form a stable hydrophobic matrix. Such reagents can include fluoroalkylsilane or long chain alkylsilane, such as octadecylsilane. The remaining photoresist substance can be removed and the solid support reacted with a suitable reagent, such as aminoalkyl silane or hydroxyalkyl silane, to form hydrophilic regions.

In some embodiments, substrate 14 can be first reacted with a suitable derivatizing reagent to form a hydrophobic surface. Such reagents can include vapor or liquid treatment of fluoroalkylsiloxane or alkylsilane. The hydrophobic surface can then be coated with a photoresist substance, photopatterned, and developed. In some embodiments, the exposed hydrophobic surface can be reacted with suitable derivatizing reagents to form hydrophilic sites. For example, the exposed hydrophobic surface can be removed by wet or dry etch such as oxygen plasma and then derivatized by aminoalkylsilane or hydroxylalkylsilane treatment. The photoresist coat can be removed to expose the underlying hydrophobic sites.

In some embodiments, substrate 14 can be first reacted with a suitable derivatizing reagent to form a hydrophilic surface. Suitable reagents can include vapor or liquid treatment of aminoalkylsilane or hydroxylalkylsilane. The derivatized surface can be coated with a photoresist substance, photopatterned, and developed. The exposed surface can be reacted with suitable derivatizing reagents to form hydrophobic sites. For example, the hydrophobic regions can be formed by fluoroalkylsiloxane or alkylsilane treatment. The photoresist coat can be removed to expose the underlying hydrophilic sites. A variety of photoresist substances and treatments useful herein can be found in the art and examples of such treatments include optical positive photoresist substances (such as, for example, AZ 1350, Novolac, marketed by Hoechst Celanese) and E-beam positive photoresist substances (such as, for example, EB-9™, polymethacrylate, marketed by Hoya Corporation, San Jose, Calif., USA).

In some embodiments, fluoroalkylsilane or alkylsilane can be employed to form a hydrophobic surface and aminoalkyl silane or hydroxyalkyl silane can be employed to form hydrophilic sites on substrate 14. Siloxane derivatizing reagents useful in forming hydrophilic sites can include, but are not limited to, those selected from: hydroxyalkyl siloxanes, such as, alkyl trichlorochlorosilane, and 7-oct-l-enyl trichlorochlorosilane; diol (bis-hydroxyalkyl) siloxanes; glycidyl trimethoxysilanes; aminoalkyl siloxanes, such as 3-aminopropyl trimethoxysilane; dimeric secondary aminoalkyl siloxanes, such as bis(3-trimethoxysilylpropyl)amine; and combinations thereof.

In some embodiments, substrate 14 for use in a surface tension array can comprise glass. Such arrays using substrate 14 comprising glass can be patterned using numerous techniques developed by the semiconductor industry using thick films (from about 1 to about 5 microns) of photoresists to generate masked patterns of exposed surfaces. After sufficient cleaning, such as by treatment with O₂ radical (such as, for example, using an O₂ plasma etch, ozone plasma treatment) followed by acid wash, first surface 11 of substrate 14 comprising glass can be derivatized with a suitable reagent to form a hydrophilic surface. In some embodiments, first surface 11 of substrate 14 comprising glass can be uniformly aminosilylated with an aminosilane, such as aminobutyidimethylmethoxysilane (DMABS). The derivatized first surface 11 of substrate 14 comprising glass can then be coated with a photoresist substance, soft-baked, photopatterned using a generic photomask to define the array patterns by exposing them to light, and developed. The underlying hydrophilic sites can be exposed in the mask area and ready to be derivatized again to form hydrophobic sites, while the photoresist coat covering region protects the underlying hydrophilic sites from further derivatization. Suitable reagents, such as fluoroalkylsilane or long chain alkylsilane, can be employed to form hydrophobic areas. For example, the exposed hydrophilic sites can be burned out with an O₂ plasma etch. The exposed regions can then be fluorosilylated. Following the hydrophobic derivatization, the remaining photoresist coat can be removed, for example by dissolution in warm organic solvents such as, methyl isobutyl ketone or N-methyl pyrrolidone (NMP), to expose the hydrophilic sites of first surface 11 of substrate 14 comprising glass. For example, the remaining photoresist can be dissolved off with sonication in acetone and then washed off in hot NMP.

In some embodiments, a surface tension array can be made without the use of photoresist. In some embodiments, first surface 11 of substrate 14 can be first reacted with a reagent to form hydrophilic sites. Certain of the hydrophilic sites can be protected with a suitable protecting agent. The remaining, unprotected, hydrophilic sites can be reacted with a reagent to form hydrophobic sites. The protected hydrophilic sites can then be deprotected. In some embodiments, first surface 11 of substrate 14 comprising glass can be first reacted with a reagent to generate free hydroxyl or amino sites. These hydrophilic sites can be reacted with a protected nucleotide coupling reagent or a linker to protect selected hydroxyl or amino sites. Suitable nucleotide coupling reagents can include, for example, a DMT-protected nucleotide phosphoramidite, and DMT-protected H-phosphonate. The unprotected hydroxyl or amino sites can then be reacted with a reagent, for example, perfluoroalkanoyl halide, to form hydrophobic sites. The protected hydrophilic sites can then be deprotected. Examples of removal of protecting groups, as well as methods useful herein, can be found in commonly assigned U.S. Pat. Nos. 6,664,388 and 6,835,827.

In some embodiments, methods provide attachment of polynucleotides to the at least one of the plurality of reaction spots 10 using an amphiphilic polymer to immobilize polynucleotides to substrate 14. In some embodiments, microplate 12 comprises an amphiphilic polymeric enhanced reaction surface which comprises a physical or chemical modification of first surface 11 of substrate 14 so as to enhance support of at least one amplification reagent. In some embodiments, an amphiphilic polymer comprises hydrocarbon backbone that can be hydrophobic in nature and comprises at least one hydrophilic moiety. Examples of a useful amphiphilic polymer can include, but are not limited to, polyvinylalcohol, polyvinylchloride, polyalkylamine, polyvinylamine, surfactants, block copolymers, dendrimers, and combinations thereof. Such modifications can include chemical treatment of first surface 11 or coating first surface 11. In some embodiments, such chemical treatment comprises chemical treatment or modification of first surface 11 so as to form hydrophilic and hydrophobic areas.

In some embodiments, 0.001% to 0.5% (% wt) solution of polyvinylalcohol (PVA) can be applied onto hydrophobic first surface 11 of substrate 14 employing a spotting method such as, for example, a pin-based fluid transfer or a piezo-based inkjet dispenser system. PVA can be an atatic material and exhibit crystallinity as the hydroxyl groups can be small enough to fit into the lattice without disrupting it. In some embodiments, PVA can have a glass transition temperature (Tg) of about 85° C. and a melting temperature (T_{M}) of about 258° C. It has been suggested that a driving force for PVA adsorption onto a hydrophobic surface can be crystallization, as discussed in, for example, Koziov et al., Macromolecules 36:16 (2003). In some embodiments, PVA film adsorbed on first surface 11 of substrate 14 creating hydrophilic regions and such PVA film can be stable at room temperatures but can be designed to dissociate as the crystalline structure melts at an elevated temperature, for example, around 100° C. In some embodiments, PVA film can be stable at room temperature but can be designed to dissociate as the crystalline structure melts at elevated temperatures such as temperatures employed in PCR cycling. In some embodiments, PVA can easily be modified due to its amphiphilic properties and can be made positively charged with amine groups which then can couple biomolecules, such as polynucleotides, either covalently or ionically.

With reference to FIG. 3, in some embodiments, an aqueous solution comprising PVA 27 can be useful in immobilizing biomolecules such as, for example, a polynucleotide at hydrophobic/water interface 28 on first surface 11 of substrate 14 since it can concentrate at hydrophobic/water interface 28 allowing adsorption and network formation to occur. In some embodiments, a biomolecule may be a polynucleotide, a protein, or a peptide. In some embodiments, PVA can adsorb irreversibly from aqueous solutions onto hydrophobic substrate 14 in contact with the aqueous solutions. In some embodiments, by lowering interfacial free, energy hydrophobic interactions or displacement of water molecules from the hydrophobic solid/water interface 28 can drive the initial steps of the adsorption of PVA onto first surface 11 of substrate 14. In some embodiments, the PVA polymer concentrates at the hydrophobic/water interface 28, exceeds a kinetic solubility in a hydrophobic region of at least one of the plurality of reaction spots 10 and crystallization ensues yielding adsorbed continuous thin films of PVA that are about 10 to about 50 Å thick. The thickness, wettability, and crystallinity of the PVA thin films depend on PVA concentration and the structure of the hydrophobic substrate 14. In some embodiments, the degree of crystallinity can be assessed using geometrical construction and a suitable calibration technique. In some embodiments, the degree of crystallinity can vary from about 10% for thin films adsorbed from 2.3 M PVA to about 30% for thin films adsorbed from 0.023 M PVA aqueous solution. In some embodiments, thinner films adsorbed from more dilute solutions can be more highly crystalline and less hydrophilic. In some embodiments, an aqueous solution comprising PVA 27 can be cross-linked at the hydrophobic/water interface 28 with glutaraldehyde, for example, in the presence of an acid. In some embodiments, highly to intermediate hydrolyzed PVA can be used to adsorb onto hydrophobic substrate 14 and immobilize biomolecules including polynucleotides. In some embodiments, cross-linking PVA can increase stability of a hydrophobic region of at least one of the plurality of reaction spots 10 in hot aqueous solutions. In some embodiments, cross-linking PVA can improve stability of hydrophobic region when performing PCR. In some embodiments, cross-linking PVA does not change hydrophilic properties of at least one of the plurality of reaction spots 10. In some embodiments, cross-linking PVA can increase the hydrophylicity of at least one of the plurality of reaction spots 10. In some embodiments, the molecular weight range of the PVA amphiphilic polymer can be between about 70,000 to about 120,000.

In some embodiments, a plurality of reaction spots 10, which can be hydrophilic, can be formed on first surface 11 of substrate by chemically treating it with compounds comprising an amphiphilic hydrocarbon, which can be activated by free amino hydroxyl, carboxyl, thiol, amido, halo, and/or sulfate moiety. In some embodiments, a plurality of reaction spots 10 can comprise a solution of PVA having a hydrophobic backbone and at least one free hydrophilic hydroxyl group available to bind a polynucleotide. In some embodiments, when PVA can be covalently attached to substrate 14, additional functional groups can be formed on the PVA polymer to conjugate a plurality of polynucleotides. In some embodiments, PVA can be activated in solution to yield at least one moiety that can bind directly to polynucleotides. In some embodiments, linking PVA to polynucleotides can occur in solution allowing immobilization of PVA-polynucleotide conjugate to substrate 14 in a one-step procedure, which can obviate a need for complicated surface conjugation procedures.

In some embodiments, the hydroxyl functional group in PVA can be used for the conjugation of biomolecules, such as, for example, polynucleotides, proteins, peptides, or capture antibodies and the like. Various conjugation chemistry methods using hydroxyl functional groups can be found in literature such as, for example, Hermann, Bioconjugate Techniques, Academic Press, San Diego, Calif. (1996). Those skilled in the art will appreciate that slight modifications in these methods may provide improved yields in conjugation. Examples of such slight modifications include use of different buffer systems and/or adjustments in the pH during conjugation.

In some embodiments, PVA can be first deposited on hydrophobic first surface 11 of substrate 14. In some embodiments, surface derivatization at least one of the plurality of reaction spots 10 enables the attachment of polynucleotides onto a desired location. Conjugation of polynucleotides on the PVA film containing hydroxyl functional group can be carried out through chemistry schemes such as surface activation or probe activation.

In some embodiments, activated functional groups of the PVA film can be introduced on first surface 11 to which a polynucleotide functional group can be conjugated onto the PVA film. In some embodiments, polynucleotide functional groups include, but are not limited to, amine and thiol. In some embodiments, surface activation can be carried out either by directly activating the hydroxyl groups on PVA or through multi-step chemistry coupling in which a different type of functional group can be introduced prior to subsequent activation. In some embodiments, hydroxyls on PVA can be activated by a cross-linker agent, in which at least one active group of the cross-linker reacts with the hydroxyl and leave the remaining active group(s) for bioconjugation with a polynucleotide. In some embodiments, cross-linking agents can include homofunctional or heterofunctional with either two (bifunctional) or multi-active groups (multi-functional). Examples of a homobifunctional cross-linker include, but not limited to, carbonyidiimidazole (CDI), N,N′-Disuccimidyl carbonate (DSC), N-Hydroxysuccimidyl chlororformate, alkyl halogens, isocyanates, epoxides, oxiranes and acyl chloride, as well as those discussed in Hermann (1996).

In some embodiments, PVA can be activated via a free hydroxyl group using cross-linking agents such as, for example, carbonyldiimidazole (CDI), N,N′-Disuccimidyl carbonate (DSC), N-Hydroxysuccimidyl chloroformate, alkyl halogens, isocyanates, epoxides, oxirane, acyl chloride, and the like. Activation of an amphiphilic polymer allows further conjugation to polynucleotides, polymers, copolymers, linkers, spacers, block polymers, dendrimers, and combinations thereof. In some embodiments, a method of conjugating a polynucleotide to PVA film can include introducing a different functional group through chemical coupling to the hydroxyl group on PVA film. For example, carboxylic acid can be introduced through reaction with anhydrides, such as, maleic anhydride, succinic anhydride, or glutaric anhydride. Upon reaction with the nucleophilic hydroxyl group of the PVA film, the ring structure of the anhydride opens and can form an acylated product modified to contain a newly formed carboxylated group. In some embodiments, carboxylic acid can also be introduced through reaction with chloroacetic acid under basic condition.

In some embodiments, upon introduction of a functional group, such as, for example, carboxylic acid or amine, the amphilic polymer (such as a PVA film) can be activated using cross-linkers that can be either of homofunctional or heterofunctional. In some embodiments, carbodiimide family, such as EDC and EDC plus sulfo-NHS, can be an effective agent for coupling of amine-terminated polynucleotide to carboxylated group. In some embodiments, a surface activation can use a homobifunctional cross-linker such as N,N′-Disuccimidyl carbonate (DSC) to react with the surface carboxylate group and leave the other NHS group for bioconjugation of a polynucleotide to an amphiphilic polymer. In some embodiments, an amphiphilic polymer can be chemically modified by adding a carboxylic acid moiety through reaction with maleic anhydride, succinic anhydride, or glutaric anhydride. In some embodiments, as illustrated in FIG. 4, a modified terminal amine containing polynucleotide 22 can be coupled using cross-linker 25 to at least one of a plurality of reaction spots 10 comprising an activated PVA.

In some embodiments, to covalently couple a polynucleotide to at least one of the plurality of reaction spots 10 can be accomplished by employing a macromolecule, such as a polymer, copolymer, block copolymer, or dendrimer, that contains at least one functional group that reacts with a hydroxyl group on a PVA film. In some embodiments, the macromolecule can be immobilized on at least one of the plurality of reaction spots 10, a subsequent polynucleotide conjugation can be carried out by either direct reaction with the remaining functional groups that do not react with the hydroxyl group or through activation of the surface functional group with a cross-linker agent. For example, a reaction of polymaleic anhydride to PVA can leave some unreacted anhydride groups for bioconjugation with an amine-terminated polynucleotide. In some embodiments, methods can include the hydrolyzation of the unreacted maleic anhydride group to form carboxylate group, which in turn can be activated using carboxylate cross-linkers, as discussed above. In some embodiments, methods to conjugate a polynucleotide on at least one of a plurality of reaction spots 10 can be through a reaction with a polynucleotide probe that contains at least one reactive functional group. In some embodiments, a polynucleotide probe can contain a reactive group such as NHS which reacts readily to a nucleophilic functional group, such as an amine group on the PVA film.

In some embodiments, PVA can be cross-linked with homobifunctional or heterobifunctional cross-linking agents such as, for example, EDC (1-Ethyl-3-[3-dimethylaminopropyl]carbodiimide Hydrochloride), DSS (Disuccinimidyl suberate), DTSSP (3,3′-Dithiobis[sulfosuccinimidylpropionate]), PMPI (N-[p-Maleimidophenyl]isocyanate) and EDP (3-[(2-Aminoethyl)dithio]propionic acid.HCl). Such homobifunctional or heterobifunctional cross-linking agents can be commercially available through Pierce, Rockford, Ill., USA and Sigma-Aldrich Corp., St. Louis, Mo., USA. In some embodiments, surface activation of PVA with an amine, a thiol, or another functional group generally can depend on the nature of a chemically modified polynucleotide to be immobilized on substrate 14.

In some embodiments, a solution of the PVA can be deposited on first surface 11 in a pattern or array, forming a plurality of reaction spots 10. Suitable materials for substrate 14 include glass such as, for example, borosilicate, flint glass, crown glass, float glass, fused silica, or high temperature plastics such, as for example, polycarbonate, polyolefins, polytetrafluoroethylene, polyetherketone, polyamideimide, polypropylene, polydimethyl siloxane, and combinations thereof. In some embodiments, PVA and polynucleotide 22 can be attached to at least one of a plurality of reaction spots 10 by the hydrophobic hydrocarbon backbone of PVA of the plurality of reaction spots 10.

In some embodiments, a chemically modified polynucleotide can be directly coupled to an activated amphiphilic polymer on substrate 14. Examples of chemically modified polynucleotides can include polynucleotides modified with amino, thiol, carboxyl and acridite moieties and such examples can be found and can be commercially available from Integrated DNA Technologies, Inc., Coralville, Iowa, USA and Glen Research, Sterling, Va., USA. In some embodiments, an aminated or carboxylated polynucleotide can be covalently immobilized to the carboxylated or aminated amphiphilic polymer via amide bonds by 1-ethyl-3-(3-dimethylaminopropyl)carbodiimide hydrochloride (EDC)-catalyzed amidation reaction.

In some embodiments, a polynucleotide can be covalently attached to a macromolecule that can be heterobifunctional. In some embodiments, a heterobifunctional macromolecule and variants thereof, refer to spacers, linkers, polymers, hydrocarbons, polyolefins, co-polymers, block copolymers, dendrimers, and the like, which can be of variable length, and possess functional groups capable of a reaction with at least two chemically distinct functional groups such as, for example, amines and thiols. In some embodiments, a heterobifunctional macromolecule can bind to one functional group present on an amphiphilic polymer such as, for example, PVA, concomitantly with a different functional group present on a polynucleotide. In some embodiments, a terminal nucleotide can be coupled to a spacer/linker phosphoramidite. In some embodiments, a spacer/linker can be a hexaethyleneglycol spacer. In some embodiments, a hydroxyl group present on PVA can be activated using an anhydride or chloroacetic acid under basic conditions. In some embodiments, a reactive carboxyl group on PVA can be linked to a polynucleotide-spacer via a cross-linking agent such as, for example, EDC, and Sulfo-NHS, which can react with a carboxyl and/or amine group to form a stable amide bond. In some embodiments, a cleavable site can be made available for a PCR protocol for cleavage of a polynucleotide 22 from at least one of a plurality of reaction spots 10. In some embodiments, an activated amphiphilic polymer can be coupled with a polynucleotide or a polynucleotide-linker molecule by using a cross-linking agent which incorporates a cleavable disulphide bond with dithiothreitol. An example of such a cross-linking agent can include AEDP (3-[2-Aminoethyl)dithio]propionic acid-HCl). In some embodiments, a linker and a polynucleotide containing a terminal reactive amine group can be immobilized reversibly with AEDP onto an amphiphilic polymer on substrate 14. In some embodiments, activation of PVA with a cross-linking agent, a forming free amino, a hydroxyl, a carboxyl, a thiol, an amido, a halo, or a sulfate group of a hydrophilic site can be covalently coupled with a linker moiety (such as, for example, polylysine, hexethylene glycol, and polyethylene glycol).

In some embodiments, a surface tension array can be made by first reacting substrate 14 with a reagent to form hydrophilic sites that can be a plurality of reaction spots 10. Some of the hydrophilic sites can be protected with a suitable protecting agent. Any unprotected hydrophilic sites can be reacted with a reagent to form hydrophobic sites. The protected hydrophilic sites can be deprotected. In some embodiments, a glass surface can be first reacted with a PVA solution to generate free hydroxyl sites. These hydroxyl sites can be reacted with a protected nucleotide coupling reagent or a linker to protect selected hydroxyl sites. Examples of suitable nucleotide coupling reagents include, for example, a DMT-protected nucleotide phosphoramidite, and DMT-protected H-phosphonate. The unprotected hydroxyl sites can then be reacted with a reagent, for example, perfluoroalkanoyl halide, to form hydrophobic sites. The protected hydrophilic sites can then be deprotected.

In some embodiments, PVA can be functionalized by a monosuccinate group then coupled to a polynucleotide such as, for example, the use of monosuccinate to functionalize PVA can be found in Sanchez-Chaves, Polymer 39:13 (1998). In some embodiments, a polynucleotide can be first attached to PVA in a solution and the resulting bioconjugates subsequently can adsorb onto hydrophobic first surface 11 of substrate 14 to create a plurality of reaction spots 10. In some embodiments, the polynucleotide attached to PVA in a solution can be deposited on hydrophobic first surface 11 of substrate 14 in one single step without prepatterning of first surface 11 to create a plurality of reaction spots 10.

In some embodiments, a chemical modality comprises chemical treatment or modification of substrate 14 so as to anchor at least one amplification reagent to the substrate 14. In some embodiments, the amplification reagent can be affixed to substrate 14 so as form an immobilization array of a plurality of reaction spots 10. In some embodiments, an anchor can be an attachment of an amplification reagent to first surface 11 of substrate 14, directly or indirectly, so that the amplification reagent can be available for a reaction such as, for example, amplification, but cannot be removed or otherwise displaced from first surface 11 of substrate 14 surface prior to the reaction during routine handling of microplate 12 and any sample preparation prior to the reaction. In some embodiments, an amplification reagent can be anchored by covalent or non-covalent bonding directly to first surface 11 of substrate 14. In some embodiments, an amplification reagent can be bonded, anchored, or tethered to an immobilization moiety which, in turn, can be anchored to the first surface 11 of substrate 14. In some embodiments, an amplification reagent can be anchored to the first surface 11 of substrate 14 through a chemically releasable or cleavable site, for example, by bonding to an immobilization moiety with a releasable site. In some embodiments, an amplification reagent can be released from substrate 14 upon reacting with cleaving reagents prior to, during or after microplate 12. Examples of such release methods include a variety of enzymatic, or non-enzymatic means, such as chemical, thermal, or photolytic treatment.

In some embodiments, suitable cleavable sites can include, but are not limited to, the following: base-cleavable sites such as esters, particularly succinates (cleavable with, for example, ammonia or trimethylamine); quaternary ammonium salts (cleavable with, for example, diisopropylamine) and urethanes (cleavable with, for example, aqueous sodium hydroxide); acid-cleavable sites, such benzylalcohol derivatives (cleavable with, for example, using trifluoroacetic acid), teicoplanin aglycone (cleavable with, for example, trifluoroacetic acid followed by base), acetals and thioacetals (cleavable with, for example, trifluoroacetic acid), thioethers (cleavable with, for example, HF or cresol) and sulfonyls (cleavable with, for example, trifluoromethane sulfonic acid, trifluoroacetic acid, thioanisole, or the like); nucleophile-cleavable sites such as phthalamide (cleavable with, for example, substituted hydrazines), esters (cleavable with, for example, aluminum trichloride) and Weinreb amide (cleavable with, for example, lithium aluminum hydride); and other types of chemically cleavable sites, including phosphorothioate (cleavable with, for example, silver or mercuric ions) and diisopropyldialkoxysilyl (cleavable with, for example, fluoride ion).

In some embodiments, an amplification reagent comprises a primer, which can be released from substrate 14 during a method of these teachings. In some embodiments, a primer can be initially hybridized to a polynucleotide immobilization moiety, and subsequently released by strand separation from an array-immobilized polynucleotide upon microplate 12 assembly. In some embodiments, a primer can be covalently immobilized on substrate 14 via a cleavable site and released before, during, or after assembly of microplate 12. For example, an immobilization moiety can comprise a cleavable site and a primer sequence. The primer sequence can be released via selective cleavage of a cleavable site before, during, or after assembly. In some embodiments, an immobilization moiety can be a polynucleotide which contains one or more cleavable sites and one or more primers. In some embodiments, a cleavable site can be introduced in an immobilized moiety during in situ synthesis. Alternatively, an immobilized moiety containing a releasable site can be prepared before covalently or non-covalently immobilizing it on substrate 14. Examples of chemical moieties for immobilization attachment to solid support include, but not limited to, those comprising carbamate, ester, amide, thiolester, (N)-functionalized thiourea, functionalized maleimide, amino, disulfide, amide, hydrazone, streptavidin, avidin/biotin, and gold-sulfide groups.

In some embodiments, as illustrated in FIG. 5, at least one of the plurality of reaction spots 10 array comprises at least one PVA network 41 bonded to substrate 14. In some embodiments, PVA network 41 can be bonded to first surface 11 of substrate 14. Substrate 14 can comprise glass such as, for example, borosilicate, flint glass, crown glass, float glass, fused silica, or high temperature plastics such as, for example, polycarbonate, polytetrafluoroethylene, polyetherketone, polyamideimide, polypropylene, polydimethyl siloxane, and combinations thereof. In some embodiments, PVA network 41 can then be synthesized with cleavable linker 33 such as, for example, a disulfide bond, then can be followed by polynucleotide 22. In some embodiments, an amplification reagent comprises a cleavable reagent 38, such as, for example, dithiothreitol that can operably cleave cleavable linker 33 thereby releasing polynucleotide 22 for use in an amplification reaction.

In some embodiments, the conjugation chemistry discussed above for attaching polynucleotide 22 to PVA can be directly applicable to single step spotting methods in solution conjugation. In some embodiments, conjugation chemistry can be more efficient in solution than on solid surface. Those skilled in the art will appreciate that care should be taken to ensure that a proper ratio of polynucleotide 22 to PVA and a proper space linker between the polynucleotide 22 and PVA are selected such as to maintain the physical properties of PVA with respect to its adsorption onto hydrophobic first surface 11 and the biological functionalities of polynucleotide 22. Those skilled in the art will appreciate that any biomolecule can be conjugated to PVA and can be directly applicable to single step spotting methods. Examples of such a biomolecule may include a polynucleotide, a protein, a peptide, or an antibody and the like, as discussed above.

In some embodiments, different species of PVA can be created such that each PVA has only one type of polynucleotide 22 attached. In some embodiments, mixture of such polynucleotide 22 modified PVA can be deposited simultaneously in one step spotting method for multiple polynucleotide immobilization on at least one of a plurality of reaction spots 10. In some embodiments, the multiple polynucleotide can be, for example, primers, detection probes, hybridization sites, targets, ligation sites, probes, and amplification reagents. In some embodiments, the ratio of each polynucleotide modified PVA can be precisely controlled. In some embodiments, this method can circumvent some of the difficulties in immobilizing multiple probes at one location on the surface. In some embodiments, a one step spotting method can be carried out with any one of a various spotting/printing techniques as discussed herein and such as, for example, contact spotting, stamping, inkjet printing, or other non-contact printing techniques. In some embodiments, spotting methods useful herein can include those disclosed in commonly assigned U.S. Pat. Nos. 6,296,702; 6,413,586; 6,440,217; 6,467,700; 6,579,367; and 6,849,127.

In some embodiments, an immobilization reagent array comprises a hydrogel affixed to the first surface 11 of substrate 14. Hydrogels useful can include those selected from cellulose gels, such as agarose and derivatized agarose; xanthan gels; synthetic hydrophilic polymers, such as, cross-linked polyethylene glycol, polydimethyl acrylamide, polyacrylamide, polyacrylic acid (such as, for example, cross-linked with dysfunctional monomers or radiation cross-linking), and micellar networks; and mixtures thereof. Derivatized agarose can include agarose which has been chemically modified to alter its chemical or physical properties. Derivatized agarose can include low melting agarose, monoclonal anti-biotin agarose, and streptavidin derivatized agarose. In some embodiments, hydrogel comprises agarose, derivatized agarose, and mixtures thereof.

In some embodiments, a solution of the hydrogel can be deposited on first surface 11 of substrate 14 in a pattern or array, forming a plurality of reaction spots 10. In some embodiments, substrate 14 can be glass such as, for example, borosilicate, flint glass, crown glass, float glass, fused silica, or high temperature plastics such as, for example, polycarbonate, polyolefins, polytetrafluoroethylene, polyetherketone, polyamideimide, polypropylene, polydimethyl siloxane, and combinations thereof. In some embodiments, as illustrated in FIG. 6, agarose fibers 20 can be mixed with agarose anti-biotin 21 and a biotinylated polynucleotide 22 such as, for example, a primer, a detection probe, a hybridization site, a ligation site, target, probe, or other amplification reagents. In some embodiments, first surface 11 of the substrate 14 can be treated with APTES or polylysine to make it have positive charge 24. In some embodiments, the natural negatively charged agarose fibers 20 comprising biotinylated polynucleotide 22 can be held by the positive charge 24 on the plurality of reaction spots 10.

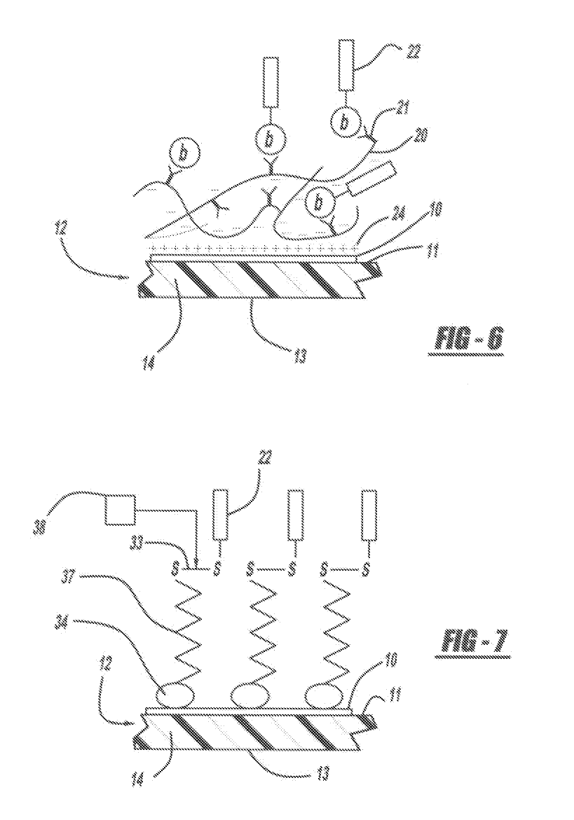

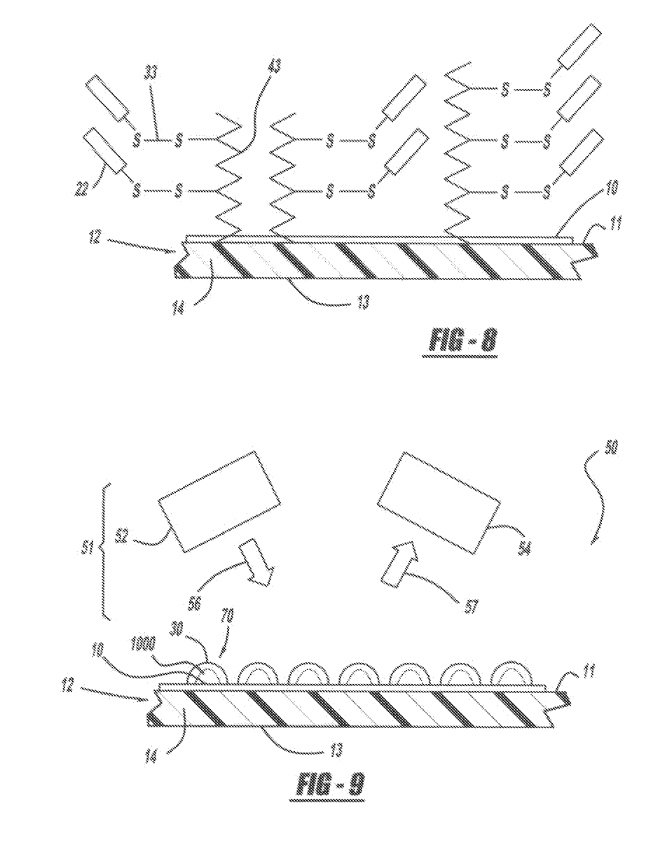

In some embodiments, as illustrated in FIG. 7, an immobilized reagent array comprises at least one streptavidin molecule 34 bonded to first surface 11 of substrate 14 forming a plurality of reaction spots 10. In some embodiments, substrate 14 can be glass such as, for example, borosilicate, flint glass, crown glass, float glass, fused silica, or high temperature plastics such as, for example, polycarbonate, polyolefins, polytetrafluoroethylene, polyetherketone, polyamideimide, polypropylene, polydimethyl siloxane, and combinations thereof. Such methods for binding streptavidin to glass can be found in, for example, Birkert, et al., Anal. Biochem., 282:200-208 (2000). In some embodiments, a streptavidin molecule 34 can be covalently bonded to first surface 11 of substrate 14. In some embodiments, polynucleotide 22, such as primer, a detection probe, a hybridization site, target, a ligation site, probe, or other amplification reagents can be attached through a cleavable linker 33 to biotin molecule 37. During a method of these teachings, a cleavable reagent 38 such as, for example, dithio threitol, can operably cleave cleavable linker 33, thereby releasing the polynucleotide 22 for use in an amplification reaction. In some embodiments, other cleavable linkers and cleavable reagents discussed herein can be employed with the attachment and cleaving of polynucleotide 22.

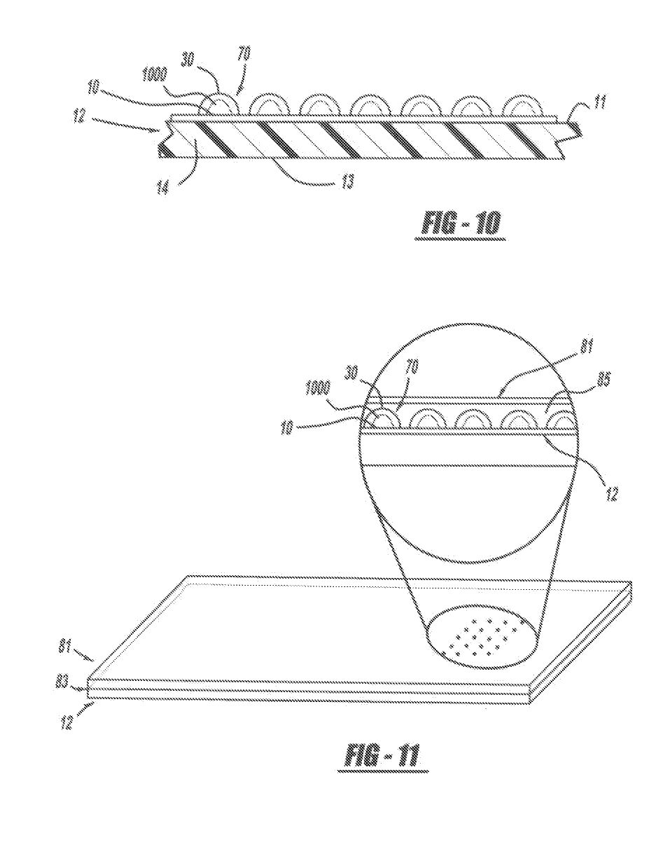

In some embodiments, as illustrated in FIG. 8, an immobilization array can comprise polyacrylamide 43 bonded to first surface 11 of substrate 14. In some embodiments, substrate 14 can comprise glass such as, for example, borosilicate, flint glass, crown glass, float glass, fused silica, or high temperature plastics, such as, for example, polycarbonate, polyolefins, polytetrafluoroethylene, polyetherketone, polyamideimide, polypropylene, polydimethyl siloxane, and combinations thereof. In some embodiments, an acridite labeled polynucleotide can then be synthesized to comprise an acridite followed by cleavable linker 33 such as, for example, disulfide bond, followed by polynucleotide 22 such as, for example, a primer, a detection probe, a hybridization site, a ligation site, a target, a probe, or other amplification reagents. In some embodiments, a dimethyl acrylamide monomer can be bonded to first surface 11 of substrate 14. In some embodiments, an acridite labeled polynucleotide can then be polymerized with dimethyl acrylamide monomer, in situ, thereby affixing the polynucleotide 22 to first surface 11 of substrate 14. In some embodiments, methods for immobilizing acrylamid-modified polynucleotides can be found in, for example, Rehman, et al., Nucleic Acids Res. 27:649 (1999). In some embodiments, during a method of these teachings, a reagent can comprise a cleavable reagent 38 such as, for example, dithio threitol, to cleave cleavable linker 33 such as, for example, a disulfide bond, thereby releasing polynucleotide 22 for use in an amplification and/or a hybridization reaction. In some embodiments, other cleavable linkers and cleavable reagents discussed herein can be employed with the attachment and cleaving of polynucleotide 22.

In some embodiments, methods for attaching a polystyrene chain to a polynucleotide can result in an amphiphilic molecule that can adsorb on first surface 11 of substrate 14 to form a plurality of reaction spots 10. In some embodiments, amphiphiles can be prepared through solid-phase synthesis on controlled pore glass beads (CPG) in a manner similar to conventional polynucleotide synthesis. A reagent that can be used to prepare the targeted amphiphiles can be a polystyrene phosphoramidite (Compound 1). In some embodiments, Compound 1 can be synthesized by reacting an alcohol-terminated polystyrene (M_{n,avg}=5.6×10³, PDI=1.1) with chlorophosphoramidite in anhydrous CH₂Cl₂. In some embodiments, the product can be precipitated from the reaction mixture by using anhydrous CH₃CN.

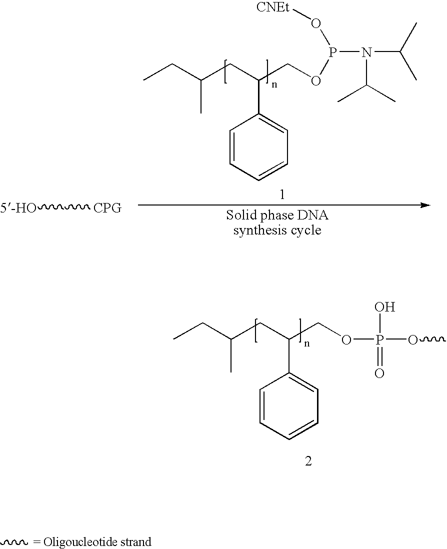

In some embodiments, Compound 1 can be used to couple a polystyrene fragment to an alcohol-terminated polynucleotide directly off the CPG. In some embodiments, the coupling of Compound 1 with the 5′ hydroxyl group of a polynucleotide strand bound to the CPG can be carried out using the syringe synthesis technique. Discussion and use of the syringe synthesis technique can be found in, for example, Storhoff, et al., J. Am. Chem. Soc. 120:1959-1964 (1998); Watson, et al., J. American Chemical Soc. 123:5592-5593 (2001); and U.S. Patent Application Publication 2003/0113740. After about 3 hours of coupling time, unreacted Compound 1 can be removed from the system by rinsing the CPG with about 50 mL of CH₂Cl₂ and about 50 mL of dimethylformamide (DMF). In some embodiments, after ammonium hydroxide deprotection and cleavage steps, the desired polystyrene-polynucleotide (Compound 2) can be soluble and can be extracted from the CPG with DMF. For example, for a 10 μmol-scale solid-phase polynucleotide synthesis, about 0.2 to about 0.4 μmol of the final amphiphile product can be collected.

In some embodiments, due to its amphiphilic nature, the polystyrene- polynucleotide conjugate (Compound 2) can form stable suspensions in various solvents including CH₂Cl₂, THF, DMF, and H₂O. Note that most polynucleotides, such as, for example, DNA, can exhibit almost no solubility in CH₂Cl₂ and THF, and polystyrene is not soluble in water. In a typical micelle formation experiment, H₂O (9 mL) can be gradually added to a DMF solution of Compound 2. The majority of the DMF can be removed from the mixture by dialysis. After dialysis, the solution can be allowed to incubate at room temperature for about 24 hours. Centrifugation can be used to remove heavily aggregated structures from the cloudy solution. This can result in a clear solution containing the micelles formed from Compound 2 as illustrated in FIGS. 12(a)-(b). Those skilled in the art will appreciate that such a method can be applicable to biomolecules other than polynucleotides. Examples of such biomolecules include proteins, peptides, or antibodies and the like. Those skilled in the art will appreciate that slight modifications may provide better yields or better purity of a polystyrene-biomolecule conjugate.

In some embodiments, a series of polystyrene-polynucleotide amphiphiles, which vary in sequence length from about less than 5 nucleotides to greater than about 25 nucleotides and vary in polystyrene molecular weight from about 4.1K to about 7.2K to about 9.5K, can yield micelle structures with tailorable average diameters from about 8 to about 30 nm. In some embodiments, these micelles can exhibit unique sequence-specific recognition properties, which derive from their hydrophilic polynucleotide shells. Examples of methods for attaching hydroxyl terminated polystyrene to a polynucleotide can be found in Zhi, et al., Nano Letters 4(6):1055-1058 (2004).

In some embodiments, phosphoramidite chemistry can be used on automated solid phase DNA synthesizer for making polystyrene attached polynucleotides using this reaction mechanism. Examples of phosphoramidite chemistry can be found in, for example, U.S. Pat. Nos. 4,415,732; 4,458,066; 4,668,777; 6,175,006; and 6,348,596. In some embodiments, as illustrated in examples of images from a microscope as shown in FIGS. 12(a)-(b), polystyrene attached polynucleotides (ps-poly) can form micelles in aqueous solutions with polystyrene core and hydrophilic polynucleotide strand on the outer layer. In some embodiments, the polystyrene moiety in ps-poly can be hydrophobic, can strongly adsorb on first surface 11 of hydrophobic substrate 14. In some embodiments, methods include one-step-spotting of ps-poly for surface immobilization on first surface 11 of hydrophobic substrate 14 based on the physical principle of hydrophobic interactions, as discussed above. In some embodiments, ps-poly can strongly adsorb on hydrophobic substrate 14, and the adhesion can withstand DNA hybridization, washing procedures, and thermocycling conditions. Examples of hydrophobic substrate 14 can include materials comprising such as, for example, borosilicate, flint glass, crown glass, float glass, fused silica, or high temperature plastics such as, for example, polycarbonate, polytetrafluoroethylene, polyetherketone, polyamideimide, polypropylene, polydimethyl siloxane, and combinations thereof.

In some embodiments, methods include multi-step spotting of ps-poly for surface immobilization on surface 11 of hydrophobic substrate 14 based on the physical principle of hydrophobic interactions, as discussed above. Multi-step spotting methods may include spotting a surface 11 of hydrophobic substrate 14 with polystyrene spots then performing surface activation on the polystyrene spots and attaching a polynucleotide to the polystyrene spot. In some embodiments, the multi-step spotting may include cross-linking the polynucleotide to the polystyrene. In some embodiments, the spotting may be a polystyrene-biomolecule conjugate. In some embodiments, the polystyrene-biomolecule conjugate may be spotted using a one-step spotting method or a multi-step spotting method. Discussion and use of attaching a biomolecule to a polystyrene surface can be found in, for example, Liu et al., Anal. Biochem., 317:76-84 (2003); and Nikiforov et al., Anal. Biochem., 227:201-209 (1995).

In some embodiments, methods of the present teachings include spotting first surface 11 of hydrophobic substrate 14 with ps-poly micelles in an aqueous solution to form a plurality of reaction spots 10. Spotting techniques are well-known in the art and can include contact printing, such as, for example, quill pin spotting; non-contact printing such as, for example, inkjet printing piezo printing; and stamping; any of these and other spotting methods known in the art. In some embodiments, manual spotting can be employed using, for example, a pipette with a volume of about 0.25 uL per reaction spot 10. In some embodiments, nanoliter droplets that form a plurality of reaction spots 10 can be printed on first surface 11 of substrate 14 using a non-contact printing instrument, such as, for example, TopSpot/E Arrayer instrument from HGS-IMIT (Freiburg, Germany). In some embodiments, a plurality of reaction spots 10 can be created using an inkjet printer. As is well-known in the art of inkjet printing, the amount of fluid that can be expelled in a single activation event of a pulse jet can be controlled by changing one or more of a number of parameters, including the orifice diameter, the orifice length (thickness of the orifice member at the orifice), the size of the deposition chamber, and the size of the heating element, among others. In some embodiments, the amount of fluid that can be expelled during a single activation event can be generally in the range about 0.1 to about 1000 pL, usually about 0.5 to about 500 pL, and more usually about 1.0 to about 250 pL. In some embodiments, a typical velocity at which the fluid can be expelled from the chamber can be more than about 1 m/s, usually more than about 10 m/s, and can be as great as about 20 m/s or greater. In some embodiments, each of the plurality of reaction spots 10 can have widths in the range from about 0.1 μm to about 1.0 cm. In some embodiments, very small reaction spots 10 sizes or feature sizes may be desired, and material can be deposited in a plurality of small reaction spots 10 whose width can be in the range about 0.1 μm to about 1.0 mm, usually about 5.0 μm to about 5000 μm, and more usually about 10 μm to 2500 μm. In some embodiments, spotting methods useful herein can include those disclosed in commonly assigned U.S. Pat. Nos. 6,296,702; 6,413,586; 6,440,217; 6,467,700; 6,579,367; and 6,849,127.

In some embodiments, microplate 12 can be covered with a sealing liquid 30 prior to the performance of analysis or reaction of assay 1000 to form reaction chamber 70, as illustrated in FIG. 10. For example, in some embodiments, sealing liquid 30 can be applied to first surface 11 of microplate 12 comprising a plurality of reaction spots 10, each comprising an assay 1000 for amplification of polynucleotide targets. In some embodiments, sealing liquid 30 can be a material which substantially covers the plurality of reaction spots 10 on microplate 12 so as to contain materials present in the plurality of reaction spots 10, and substantially prevents movement of material from one of the plurality of reaction spots 10 to another of the plurality of reaction spots 10 on substrate 14. In some embodiments, sealing liquid 30 can be any material which does not react with assay 1000 under normal storage or usage conditions such as for PCR applications and methods. In some embodiments, sealing liquid 30 can be substantially immiscible with assay 1000. In some embodiments, sealing liquid 30 can be transparent, can have a refractive index similar to or less than glass, can have low or no fluorescence, can have a low viscosity, and/or can be curable. In some embodiments, sealing liquid 30 can comprise a flowable, curable fluid such as, a curable adhesive selected from: light-curable adhesives such as, a ultra-violet-curable heat, two-part, or moisture activated adhesives; and cyanoacrylate adhesives. Such curable liquids can include, for example, Norland optical adhesives marketed by Norland Products, Inc. (New Brunswick, N.J., USA), and ocyanoacrylate adhesives such as, for example, can be found in U.S. Pat. Nos. 4,866,198 and 5,328,944, and marketed by Loctite Corporation (Newington, Conn., USA). In some embodiments, sealing liquid 30 can be selected from mineral oil, silicone oil, fluorinated oil, and other fluids which are substantially non-miscible with water. Examples of a suitable sealing liquid 30 include biological grade mineral oil marketed by Fluka (St. Louis, Mo.), mineral oil, PCR reagent marketed by Sigma-Aldich (St. Louis, Mo.), as well as CAS No. 8012-95-1, 8042-47-5. In some embodiments, sealing liquid 30 can be a fluid when it is applied to substrate 14 of microplate 12. In some embodiments, sealing liquid 30 can remain fluid throughout a reaction using microplate 12. In some embodiments, sealing liquid 30 can become a solid or semi-solid after it is applied to substrate 14 of microplate 12.

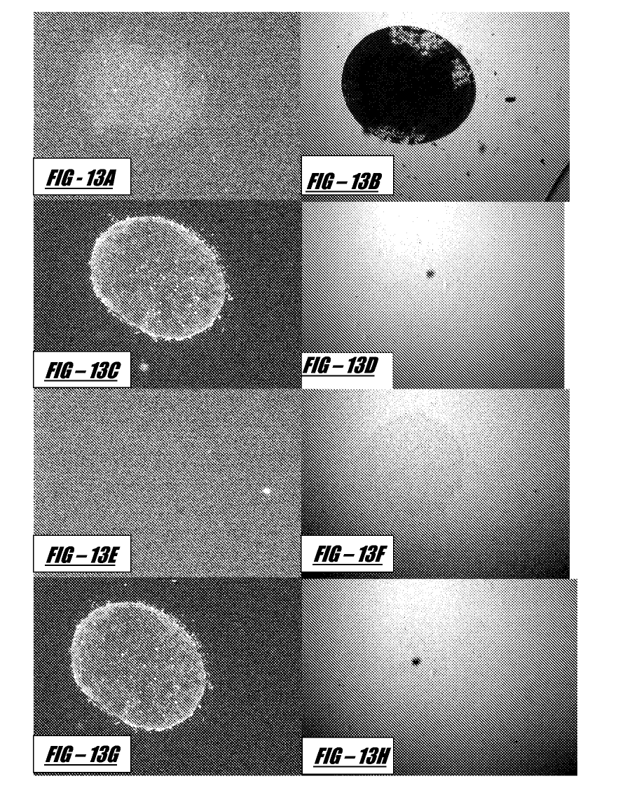

In some embodiments, as illustrated in FIG. 11, an apparatus can comprise cover 81, sealing gasket 83, and microplate 12 comprising assay 1000 on at least one of the plurality of reaction spots 10 with assay 1000 covered by sealing liquid 30. In some embodiments, sealing gasket 83 can have a height of about 259 μm. In some embodiments, sealing gasket 83 creates volume 85 between microplate 12 and cover 81. In some embodiments, volume 85 can be filled with sealing liquid 30. In some embodiments, sealing gasket 83 can further comprise a hole, port, or valve for removing excess sample, reagents, and/or sealing liquid.

With reference to FIG. 10 and FIG. 11, in some embodiments, forming reaction chamber 70 can be effected by any method by which the contents of each of the plurality of reaction spots 10 are physically isolated from adjacent reaction spots. In some embodiments, physical isolates can be the creation of a barrier which substantially prevents physical transfer of reactants, (such as, for example, a polynucleotide target), amplification reagents, and amplification reaction products such as, amplicons between reaction chamber 70. Such method of physical isolation also physically isolates reaction chamber 70 from the environment such that reactants and reaction products cannot be lost to the air or to surrounding surfaces of microplate 12 through, for example, evaporation. In some embodiments, forming of reaction chamber 70 can be effected by applying sealing liquid 30 to first surface 11 of substrate 14. Such methods of applying include those described above regarding the application of reactants.

In some embodiments, microplate 12 comprises substrate 14 having at least about 10,000 reaction spots 10, each spot comprising at least one unique PCR primer and having a volume of assay 1000 of less than about 20 nanoliters (nl), as well as sealing liquid 30 covering substrate 14 and isolating each of the plurality of reaction spots 10. The density of the plurality of reaction spots 10 (i.e., number of spots per unit surface area of substrate 14), and the size and volume of the plurality of reaction spots 10, can vary depending on the desired application. In some embodiments, a density of the plurality of reaction spots 10 on substrate 14 can be from about 10 to about 10,000 spots/cm². In some embodiments, a density of the plurality of reaction spots 10 on substrate 14 can be from about 50 to about 1000 spots/cm², or from about 50 to about 600 spots/cm². In some embodiments, a density of the plurality of reaction spots 10 on substrate 14 can be from about 150 to about 170 spots/cm². In some embodiments, a density of the plurality of reaction spots 10 on substrate 14 can be from about 480 to about 500 spots/cm². In some embodiments, an area of each retention site can be from about 1.0 μm² to about 0.05 mm² or from about 2.0 μm² to about 0.04 mm². In some embodiments, a volume of assay 1000 can be retained on at least one of the plurality of reaction spots 10 can be from about 0.05 nl to about 500 nl or from about 0.1 nl to about 200 nl. In some embodiments, a volume of assay 1000 can be retained on at least one of the plurality of reaction spots 10 can be from about 1 nl to about 5 nl or about 2 nl. In some embodiments, a volume of assay 1000 can be retained on at least one of the plurality of reaction spots 10 can be less than about 2 nl. In some embodiments, a volume of assay 1000 can be retained on at least one of the plurality of reaction spots 10 can be from about 80 nl to about 120 nl. In some embodiments, a pitch of the plurality of reaction spots 10 in an array can be from about 50 μm to about 10,000 μm or from about 50 μm to about 6000 μm. In some embodiments, a pitch can be from about 4000 μm to 5000 μm or about 4500 μm.

In some embodiments, a total number of the plurality of reaction spots 10 on substrate 14 can be from about 200 to about 100,000 or from about 500 to about 50,000. In some embodiments, microplate 12 comprises from about 500 to about 10,000 reaction spots 10 or from about 1,000 to about 7,000 reaction spots 10. In some embodiments, microplate 12 comprises from about 10,000 to about 50,000 reaction spots 10, or from about 15,000 to about 40,000 reaction spots 10, or from about 20,000 to about 35,000 reaction spots 10. In some embodiments, microplate 12 comprises about 30,000 reaction spots 10.

In some embodiments, substrate 14 can comprise raised or depressed regions such as, for example, features might be barriers and trenches to aid in the distribution and flow of liquids on first surface 11 of substrate 14. The dimensions of these features are flexible, depending on factors, such as, avoidance of air bubbles upon assembly, mechanical convenience and feasibility, etc.

In some embodiments, microplate 12 can be used for the amplification of at least one polynucleotide target, such as by PCR. Briefly, by way of background, PCR can be used to amplify a sample of at least one polynucleotide target such as, for example, DNA for analysis. In some embodiments, polynucleotide targets can be derived from any organism or other source including, but not limited to, prokaryotes, eukaryotes, plants, animals, and viruses, as well as synthetic nucleic acids, for example. In some embodiments, a polynucleotide target can originate from any of a wide variety of sample types, such as cell nuclei (such as, for example, genomic DNA), whole cells, tissue samples, phage, plasmids, mitochondria, and the like. In some embodiments, a polynucleotide target can contain DNA, RNA, cDNA and/or variants or modifications thereof. Typically, the PCR reaction involves copying the strands of the at least one polynucleotide target and then using the copies to generate additional copies in subsequent cycles. Each cycle doubles the amount of the at least one polynucleotide target present, thereby resulting in a geometric progression in the number of copies of the at least one polynucleotide target. The temperature of a double-stranded polynucleotide target is elevated to denature the at least one polynucleotide target, and the temperature is then reduced to anneal one primer to each strand of the denatured at least one polynucleotide target. In some embodiments, the at least one polynucleotide target can be a cDNA, DNA, RNA, or a fragment thereof. In some embodiments, primers are used as a pair—a forward primer and a reverse primer—and can be referred to as a primer pair or primer set. In some embodiments, the primer set comprises a 5′ upstream primer that can bind with the 5′ end of one strand of at least one polynucleotide target and a 3′ downstream primer that can bind with the 3′ end of the other strand of at least one polynucleotide target. Once a given primer binds to the strand of at least one polynucleotide target, the primer can be extended by the action of a polymerase. In some embodiments, the polymerase can be a thermostable DNA polymerase, for example, a Taq polymerase. The product of this extension, which sometimes can be referred to as an amplicon, can then be denatured from the resultant strands and the process can be repeated. Temperatures suitable for carrying out the reactions are well-known in the art. Certain basic principles of PCR are set forth in U.S. Pat. Nos. 4,683,195; 4,683,202; 4,800,159; and 4,965,188, each issued to Mullis et al.

In some embodiments, a detection probe comprises a moiety that facilitates detection of a polynucleotide target, and in some embodiments, quantifiably. In some embodiments, a detection probe can comprise, for example, a fluorophore such as a fluorescent dye, a hapten such as a biotin or a digoxygenin, a radioisotope, an enzyme, or an electrophoretic mobility modifier. In some embodiments, the level of amplification can be determined using a fluorescently labeled polynucleotide. In some embodiments, a detection probe can comprise a fluorophore further comprising a fluorescence quencher. In some embodiments, a detection probe comprises a moiety that facilitates detection of a polynucleotide of interest, and in some embodiments, quantifiably.

In some embodiments, a detection probe can comprise a fluorophore and can be, for example, a 5′-exonuclease assay probe such as a TaqMan® probe (marketed by Applied Biosystems); a stem-loop molecular beacon (such as, for example, U.S. Pat. Nos. 5,925,517 and 6,103,476; Nature Biotechnology 14:303-308 (1996); Vet et al., Proc Natl Acad Sci USA 96:6394-6399 (1999)), a stemless or linear molecular beacon (such as, for example, PCT Patent Publication No. WO 99/21881), a Peptide Nucleic Acid (PNA) Molecular Beacon™ (such as, for example, U.S. Pat. Nos. 6,355,421 and 6,593,091), a linear PNA molecular beacon (such as, for example, Kubista et al., SPIE 4264:53-58 (2001)), a flap endonuclease probe (such as, for example, U.S. Pat. No. 6,150,097), a Sunrise®/Amplifluor®) probe (such as, for example, U.S. Pat. No. 6,548,250), a stem-loop and duplex Scorpion™ probe (such as, for example, Solinas et al., Nucleic Acids Research 29:E96 (2001), and U.S. Pat. No. 6,589,743), a bulge loop probe (such as, for example, U.S. Pat. No. 6,590,091), a pseudo knot probe (such as, for example, U.S. Pat. No. 6,589,250), a cyclicon (such as, for example, U.S. Pat. No. 6,383,752), an MGB Eclipse™ probe (marketed by Epoch Biosciences), a hairpin probe (such as, for example, U.S. Pat. No. 6,596,490), a PNA light-up probe, a self-assembled nanoparticle probe, or a ferrocene-modified probe described, for example, in U.S. Pat. No. 6,485,901; Mhlanga et al., Methods 25:463-471 (2001); Whitcombe et al., Nature Biotechnology 17:804-807 (1999); Isacsson et al., Molecular Cell Probes 14:321-328 (2000); Svanvik et al., Anal. Biochem. 281:26-35 (2000); Wolffs et al., Biotechniques 766:769-771 (2001); Tsourkas et al., Nucleic Acids Research 30:4208-4215 (2002); Riccelli et al., Nucleic Acids Research 30:4088-4093 (2002); Zhang et al., Sheng Wu Hua Xue Yu Sheng Wu Li Xue Bao (Shanghai), Acta Biochimica et Biophysica Sinica, 34:329-332 (2002); Maxwell et al., J. Am. Chem. Soc. 124:9606-9612 (2002); Broude et al., Trends Biotechnol. 20:249-56 (2002); Huang et al., Chem Res. Toxicol. 15:118-126 (2002); Yu et al., J. Am. Chem. Soc. 14:11155-11161 (2001). In some embodiments, a detection probe can comprise a sulfonate derivative of a fluorescent dye, a phosphoramidite form of fluorescein, or phosphoramidite forms of CY5. Detection probes among those useful herein are also disclosed, for example, in U.S. Pat. Nos. 5,188,934; 5,750,409; 5,847,162; 5,853,992; 5,936,087; 5,986,086; 6,020,481; 6,008,379; 6,130,101; 6,140,500; 6,140,494; 6,191,278; and 6,221,604. Energy transfer dyes among those useful herein include those described in U.S. Pat. Nos. 5,728,528; 5,800,996; 5,863,727; 5,945,526; 6,335,440; and 6,849745; U.S. Patent Application Publication No. 2004/0126763, PCT Publication No. WO 00/13026, PCT Publication No. WO 01/19841, U.S. Patent Application Ser. No. 60/611,119, filed Sep. 16, 2004, and U.S. patent application Ser. No. 10/788,836, filed Feb. 26, 2004. In some embodiments, a detection probe can comprise a fluorescence quencher such as a black hole quencher (marketed by Metabion International AG), an Iowa Black™ quencher (marketed by Integrated DNA Technologies), a QSY quencher (marketed by Molecular Probes, Inc.), and Dabsyl and Eclipse™ Dark Quenchers (marketed by Epoch).

In some embodiments, a detection probe can comprise a fluorescent dye. In such embodiments, the fluorescent dye can comprise at least one of rhodamine green (R110), 5-carboxyrhodamine, 6-carboxyrhodamine, N,N′-diethyl-2′,7′-dimethyl-5-carboxy-rhodamine (5-R6G), N,N′-diethyl-2′,7′-dimethyl-6-carboxyrhodamine (6-R6G), 5-carboxy-2′,4′,5′,7′,-4,7-hexachlorofluorescein, 6-carboxy-2′,4′,5′,7′,4,7-hexachloro-fluorescein, 5-carboxy-2′,7′-dicarboxy-4′,5′-dichlorofluorescein, 6-carboxy-2′,7′-dicarboxy-4′,5′-dichlorofluorescein, 5-carboxy-2′,4′,5′,7′-tetrachlorofluorescein, 1′,2′-benzo-4′-fluoro-7′,4,7-trichloro-5-carboxyfluorescein, or 1′,2′-benzo-4′-fluoro-7′,4,7-trichloro-6-carboxy-fluorescein, 1′,2′,7′,8′-dibenzo-4,7-dichloro-5-carboxyfluorescein.

In some embodiments, amplicons can be detected in double-stranded form by a detection probe comprising an intercalating or a cross-linking dye, such as ethidium bromide, acridine orange, or an oxazole derivative, for example, SYBR Green® (marketed by Molecular Probes, Inc.), which exhibits a fluorescence increase or decrease upon binding to double-stranded nucleic acids. In some embodiments, a detection probe comprises SYBR Green® or Pico Green® (marketed by Molecular Probes, Inc.).

In some embodiments, a detection probe can comprise an enzyme that can be detected using an enzyme activity assay. An enzyme activity assay can utilize a chromogenic substrate, a fluorogenic substrate, or a chemiluminescent substrate. In some embodiments, the enzyme can be an alkaline phosphatase, and the chemiluminescent substrate can be (4-methoxyspiro[1,2-dioxetane-3,2′(5′-chloro)-tricyclo[3.3.1.13,7]decan]-4-yl) phenylphosphate. In some embodiments, a chemiluminescent alkaline phosphatase substrate can be CDP-Star® chemiluminescent substrate or CSPD® chemiluminescent substrate (marketed by Applied Biosystems).

In some embodiments, the present teachings can employ any of a variety of universal detection approaches involving Real-Time PCR and related approaches. For example, the present teachings contemplate embodiments in which an encoding ligation reaction is performed in a first reaction vessel (such as, for example, an Eppendorf tube), and a plurality of decoding reactions are then performed in microplate 12 described herein. For example, a multiplexed oligonucleotide ligation reaction (OLA) can be performed to query a plurality of target DNA, so that each of the resulting reaction products is encoded with, for example, a primer portion, and/or a universal detection portion. By including a distinct primer pair in each of the plurality of reaction spots 10 of microplate 12 corresponding to the primer's sequences encoded in the OLA, a given encoded target DNA can be amplified by that distinct primer pair in a given spot of the plurality of reaction spots 10. Further, a universal detection probe (such as, for example, a nuclease cleavable TaqMan® probe) can be included in each of the plurality of reaction spots 10 of microplate 12 to provide for universal detection of a single universal detection probe. Such approaches can result in a universal microplate 12, with its attendant benefits including, among other things, one or more of economies of scale, manufacturing, and/or ease-of-use. The nature of the multiplexed encoding reaction can comprise any of a variety of techniques, including a multiplexed encoding PCR pre-amplification or a multiplexed encoding OLA. Further, various approaches for encoding a first sample with a first universal detection probe, and a second sample with a second universal detection probe, thereby allowing for two sample comparisons in a single microplate 12, can also be performed according to the present teachings. Illustrative embodiments of such encoding and decoding methods can be found, for example, in commonly known PCT Publication Nos. WO2003US0029693 and WO2003US0029967; and U.S. Provisional Application Nos. 60/556157; 60/556162; 60/556163; 60/556224; and 60/630681.

In some embodiments, PCR can be conducted under conditions allowing for quantitative and/or qualitative analysis of one or more polynucleotide targets. Accordingly, detection probes can be used for detecting the presence of at least one polynucleotide target in assay 1000. In some embodiments, detection probes can comprise physical (such as, for example, fluorescent) or chemical properties that change upon binding of the detection probe to at least one polynucleotide target. Some embodiments of the present teachings can provide real time fluorescence-based detection and analysis of amplicons as described, for example, in commonly assigned PCT Publication No. WO 95/30139, U.S. patent application Ser. No. 08/235,411 and U.S. Pat. Nos. 5,972,716; 5,928,907; and 6,015,674.

In some embodiments, assay 1000 can be a homogenous polynucleotide amplification assay, for coupled amplification and detection, in which the process of amplification generates a detectable signal and the need for subsequent sample handling and manipulation to detect the amplified product is minimized or eliminated. Homogeneous polynucleotide amplification assay can provide for amplification that is detectable without opening a sealed reaction chamber 70 or further processing steps once amplification is initiated. Such homogeneous polynucleotide amplification assays can be suitable for use in conjunction with detection probes. For example, in some embodiments, the use of a detection probe, specific for detecting a particular at least one polynucleotide target can be included in an amplification reaction in addition to a polynucleotide binding agent of the present teachings. Homogenous polynucleotide amplification assay among those useful herein are described, for example, in commonly assigned U.S. Pat. No. 6,814,934.

In some embodiments, methods are provided for detecting a plurality of polynucleotide targets. Such methods include those comprising forming an initial mixture comprising an analyte sample suspected of comprising at least one polynucleotide target, a polymerase, and a plurality of primer sets. In some embodiments, each primer set comprises a forward primer and a reverse primer and at least one detection probe unique for one of the plurality of primer sets. In some embodiments, the initial mixture can be formed under conditions in which one primer elongates if hybridized to a polynucleotide target.

In some embodiments, reagents can be provided comprising a master mix comprising at least one of catalysts, initiators, promoters, cofactors, enzymes, salts, buffering agents, chelating agents, and combinations thereof. In some embodiments, reagents can include water, a magnesium catalyst (such as MgCl2), polymerase, a buffer, and/or dNTP. In some embodiments, specific master mixes can comprise AmpliTaq® Gold PCR Master Mix, TaqMan® Universal Master Mix, TaqMan® Universal Master Mix No AmpErase® UNG, Assays-by-Design^{SM}, Pre-Developed Assay Reagents (PDAR) for gene expression, PDAR for allelic discrimination, and Assays-On-Demand®, (all of which are marketed by Applied Biosystems). However, the present teachings should not be regarded as being limited to the particular chemistries and/or detection methodologies recited herein, but can employ TaqMan®; Invader®; TaqMan Gold®; protein, peptide, and immuno assays; receptor binding; enzyme detection; and other screening and analytical methodologies.

In some embodiments, a method comprises performing PCR on a polynucleotide target in a complex mixture of polynucleotides. In some embodiments, a method comprises simultaneously amplifying a plurality of polynucleotide targets in a complex mixture of polynucleotides in which simultaneously amplifying can be the conducting amplification of two or more polynucleotide targets in a single mixture of polynucleotides at substantially the same time. In some embodiments, each of the polynucleotide targets can be simultaneously amplified in each of the plurality of reaction chambers 70.

In some embodiments, a method can be conducted on microplate 12 containing plurality of reaction spots 10, where each of the plurality of reaction spots 10 comprises reagents for amplifying a single polynucleotide target. In some embodiments, each of the plurality of reaction spots 10 comprises reagents for amplifying one or more polynucleotide targets that are distinct from a polynucleotide target to be amplified in other of the plurality of reaction spots 10 on microplate 12. In some embodiments, microplate 12 comprises a plurality of reaction spots 10 comprising reagents for amplifying the same individual or group of polynucleotide targets.

In some embodiments, microplate 12 can be used for analysis of polynucleotides comprising or derived from genetic materials from organisms. In some embodiments, such materials comprise or are derived from substantially the entire genome of an organism. In some embodiments, such organisms include, for example, humans, mammals, mice, Arabidopsis or any other plant, bacteria, fungi, or animal species. In some embodiments, assay 1000 comprises at least one of a homogenous solution of at least one polynucleotide target, at least one primer set for detection of at least one polynucleotide target comprising or derived from such genetic materials, at least one detection probe, a polymerase, and a buffer. In some embodiments, assay 1000 comprises at least one of a plurality of different detection probes and/or primer sets to perform multiplex PCR, which can be particularly useful when analyzing a whole genome having, for example, about 30,000 different genes. In some embodiments, analysis of substantially the entire genome of an organism can be conducted on a single microplate 12, or on multiple microplates 12 (such as, for example, two, three, four or more) each comprising subparts of such materials comprising or derived from the genetic materials of the organism. In some embodiments using multiple microplates, a plurality of microplates 12 can contain a plurality of assay 1000 having essentially identical materials or a plurality of assay 1000 having different materials. In some embodiments, a plurality of microplates do not contain assay 1000 having essentially identical materials. In some embodiments, microplate 12 comprises a fixed subset of a genome. It should also be recognized that the present teachings can be used in connection with genotyping, gene expression, or other analysis.

In some embodiments, microplate 12 comprises an alignment feature such as, for example, a corner chamfer, a pin, a slot, a cut corner, an indentation, a graphic, or other unique feature that is capable of interfacing with a corresponding feature formed in a fixture, reagent dispensing equipment, and/or thermocycler. In some embodiments, an alignment feature comprises a nub or protrusion.

In some embodiments, microplate 12 comprises marking indicia, such as graphics, printing, lithograph, pictorial representations, symbols, bar codes, handwritings or any other type of writing, drawings, etchings, indentations, embossments or raised marks, machine readable codes (i.e. bar codes, etc.), text, logos, colors, and the like. In some embodiments, marking indicia is permanent.

In some embodiments, marking indicia can be printed upon microplate 12 using any known printing system, such as, for example, inkjet printing, pad printing, hot stamping, and the like. In some embodiments, such as those using a light-colored microplate 12, a dark ink can be used to create marking indicia or vice versa.

In some embodiments, microplate 12 can be made of plastic and have a surface treatment applied thereto to facilitate applying marking indicia. In some embodiments, such surface treatment comprises flame treatment, corona treatment, treating with a surface primer, or acid washing. However, in some embodiments, a UV-curable ink can be used for printing on microplates comprising plastic.

Still further, in some embodiments, marking indicia can be printed upon microplate 12 using a CO₂ laser marking system. Laser marking systems evaporate material from a surface of microplate 12. Because CO₂ laser etching can produce reduced color changes of marking indicia relative to the remaining portions of microplate 12, in some embodiments, a YAG laser system can be used to provide improved contrast and reduced material deformation.

In some embodiments, a laser activated pigment can be added to the material used to form microplate 12 to obtain improved contrast between marking indicia and substrate 14. In some embodiments, an antimony-doped tin oxide pigment can be used, which is easily dispersed in polymers and has marking speeds as high as 190 inches per second. Antimony-doped tin oxide pigments can absorb laser light and can convert laser energy to thermal energy in embodiments where indicia are created using a YAG laser.

In some embodiments, marking indicia can identify microplate 12 to facilitate identification during processing. Furthermore, in some embodiments, marking indicia can facilitate data collection so that microplate 12 can be positively identified to properly correlate acquired data with the corresponding assay. Such marking indicia can be employed as part of Good Laboratory Practices (GLP) and Good Manufacturing Practices (GMP), and can further, in some circumstances, reduce labor associated with manually applying adhesive labels, manually tracking microplates, and correlating data associated with a particular microplate.

In some embodiments, marking indicia can assist in alignment by placing a symbol or other machine-readable graphic on microplate 12. An optical sensor or optical eye can detect marking indicia and can determine a location of microplate 12. In some embodiments, such location of microplate 12 can then be adjusted by thermocycler system 50 to achieve a predetermined position.

In some embodiments, a radio frequency identification (RFID) tag can be used to electronically identify microplate 12. RFID tag can be attached or molded within microplate 12. An RFID reader can be integrated into thermocycler system 50 to automatically read a unique identification and/or data handling parameters of microplate 12. Further, RFID tag does not require line-of-sight for readability.

In some embodiments, the location of a fluorescent signal on a solid support, such as microplate 12, can be indicative of the identity of a polynucleotide target comprised by assay 1000. In some embodiments, a plurality of detection probes can be distributed to identify loci of at least some of the plurality of reaction spots 10 of microplate 12. A signal deriving from a detection probe such as, for example, an increase in fluorescence intensity of a fluorophore at a particular locus can be detected if an amplification product binds to a detection probe and is then amplified. The location of the locus can indicate the identity of at least one polynucleotide target, and the intensity of the fluorescence can indicate the quantity of at least one polynucleotide target.

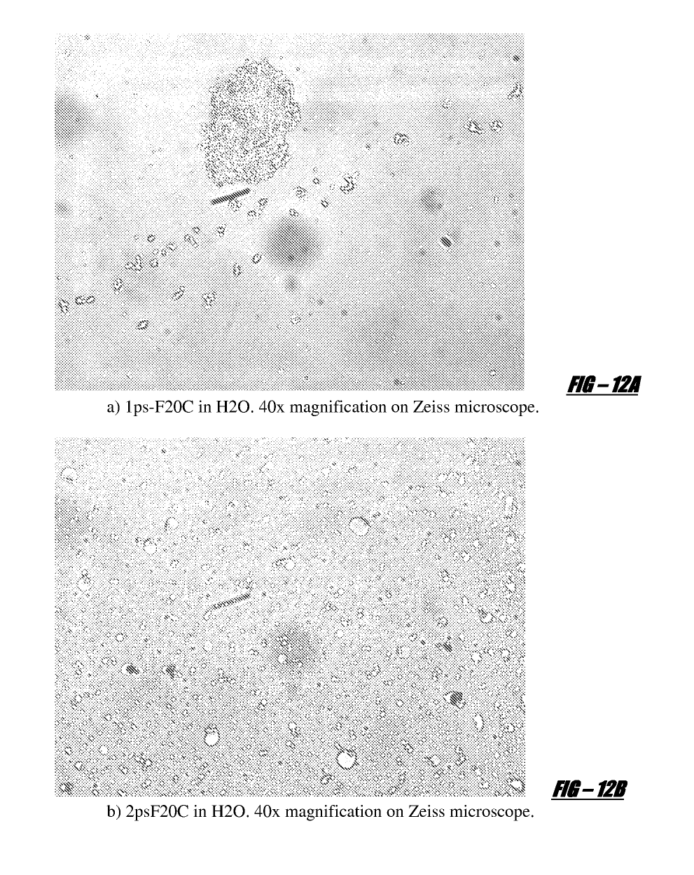

In some embodiments, methods can be performed with equipment which aids in one or more steps of amplification including handling of the microplates, thermocycling, and detection. In some embodiments, as illustrated in FIG. 9, thermocycler system 50 comprises thermocycler block 60 for supporting microplate 12, and optical system 51 comprising at least excitation source 52 for illuminating assay 1000 in at least one of the plurality of reaction chambers 70, and detection system 54.

In some embodiments, thermocycler system 50 comprises at least one thermocycler block 60. Thermocycler system 50 provides heat transfer between thermocycler block 60 and microplate 12 during analysis to vary the temperature of assay 1000. It should be appreciated that, in some embodiments, thermocycler block 60 can also provide thermal uniformity across microplate 12 to facilitate accurate and precise quantification of an amplification reaction. In some embodiments, a control system (not shown) can be operably coupled to thermocycler block 60 to output a control signal to regulate a desired thermal output of thermocycler block 60. In some embodiments, the control signal of control system can be varied in response to an input from a temperature sensor.

In some embodiments, thermocycler block 60 comprises a plurality of fin members disposed along a side thereof to dissipate heat. In some embodiments, thermocycler block 60 comprises at least one of a forced convection temperature system that blows hot and cool air onto microplate 12; a system for circulating heated and/or cooled gas or fluid through channels in microplate 12; a Peltier thermoelectric device; a refrigerator; a microwave heating device; an infrared heater; or any combination thereof. In some embodiments, thermocycler system 50 comprises a heating or cooling source in thermal connection with a heat sink. In some embodiments, the heat sink can be configured to be in thermal communication with microplate 12. In some embodiments, thermocycler block 60 continuously cycles the temperature of microplate 12. In some embodiments, thermocycler block 60 cycles and then holds the temperature for a predetermined amount of time. In some embodiments, thermocycler block 60 maintains a generally constant temperature for performing isothermal reactions such as, for example, isothermal amplification reactions upon or within microplate 12.

In some embodiments, thermocycler system 50 comprises temperature control mechanisms, for example, force convection temperature control mechanisms. Such mechanisms can be found in the art and can include, for example, those described in commonly assigned U.S. Pat. Nos. 5,928,907 and 5,942,432. Temperature control mechanisms can be included to change the temperature of microplate 12 so as to change the temperature of assay 1000 placed in at least one of the plurality of reaction chambers 70. For example, thermocycling of the assay 1000 can be desirable, particularly in methods for performing PCR or similar amplification reactions.

In some embodiments, as generally illustrated in FIG. 9, thermocycler system 50 comprises optical system 51 which comprises excitation source 52 and detection system 54. In some embodiments, excitation source 52 provides excitation light 56 comprising radiant energy of proper wavelength so as to allow detection of at least one detection probe in at least one of the plurality of reaction chambers 70. Depending on detection probes used, excitation source 52 can emit excitation light 56 that can be visible or non-visible wavelengths including, for example, infrared, visible, or ultraviolet light. In some embodiments, excitation source 52 provides excitation light 56 that excites a fluorophore in a detection probe. In some embodiments, excitation source 52 can be selected to emit excitation light 56 at one or several wavelengths or wavelength ranges.

In some embodiments, excitation light source 52 can direct excitation light 56 to each of the plurality of reaction chambers 70. In some embodiments, excitation source 52 can direct excitation light in a sequential manner to each of the reaction chambers 70 and can employ a laser and a plurality of lenses which can linearly translate in a first direction relative to microplate 12. A plurality of lenses, microplate 12, or a combination of the two can be moved, so that a relative motion is imparted between a plurality of lenses and microplate 12. In some embodiments, excitation source 52 comprises a laser emitting excitation light 56 of a wavelength of about 488 nm. In some embodiments, excitation source 52 comprises a halogen lamp. In some embodiments, excitation source 52 comprises a plurality of LED sources. In some embodiments, excitation light 56 from excitation source 52 can be directed to at least one of plurality of reaction chambers 70 in any suitable manner, for example, by employing lens, filters, mirrors, wave guides, and other optical components known in the art, as well as combinations thereof. In some embodiments, the excitation light 56 can be directed to a lens by using one or more mirrors to reflect the excitation light 56 at a desired lens. In some embodiments, the excitation light 56 can be directed to substantially all of the plurality of reaction chambers 70 simultaneously. After the excitation light 56 passes onto at least one of the plurality of reaction chambers 70, a detection probe in the at least one of the plurality of reaction chambers 70 can be illuminated, thereby emitting emission light 57. The emission light 57 can then be detected by detection system 54.

In some embodiments, detection system 54 can analyze emission light 57 from the at least one of the plurality of reaction chambers 70. In some embodiments with a single wavelength light processing element, detection system 54 can be limited to analyzing emission light 57 of a single wavelength, thereby one or more detection systems 54 each having a single detection element can be provided. In some embodiments, detection system 54 can further include a light detection device for analyzing emission light 57 from assay 1000 for its spectral components. In some embodiments, detection system 54 comprises a multi-element photodetector which can analyze emission light 57 that comprise many wavelengths. Examples of multi-element photodetectors include, but are not limited to, charge-coupled devices (CCDs), diode arrays, photo-multiplier tube arrays, charge-injection devices (CIDs), CMOS detectors, and avalanche photodiodes. In some embodiments, a multi-element photodetector can collect a single wavelength of emission light 57 simultaneously from substantially all of the reaction chambers 70 on microplate 12. In some embodiments, the detector can include a shutter and, in some embodiments, the detector can calibrate for dark current when the shutter is closed. In some embodiments, the detector system 54 includes a filter that can be placed in front of a detector to block an unwanted wavelength from entering the detector. In some embodiments, the filter can be part of a filter wheel, which comprises a plurality of filters, which can be moved in front of the detector. In some embodiments, with a filter wheel, the microplate 12 can be scanned a number of times, each time with a different filter. In some embodiments, the multi-element photodetector can be a CCD. In some embodiments, detection system 54 can be a single element detector. With a single element detector, each of reaction chambers 70 can be read one at a time. In some embodiments, the emission light 57 from substantially all of the plurality of reaction chambers 70 can be detected simultaneously such as, for example, by use of a CCD as the detector. A detector system 54 can be used in combination with a filter wheel (not shown). Examples of single dimensional detectors include, but are not limited to, one-dimensional line scan CCDs, and single photo-multiplier tubes, where the single dimension can be used for either spatial or spectral separation. It will be understood that several single dimension detectors can be used in combination with a dichroic beam splitter. In some embodiments, optical system 51 comprises a light separating element such as a light dispersing element. Light dispersing elements comprise elements that separate light into its spectral components, such as transmission gratings, reflective gratings, prisms, and combinations thereof. Other light separating elements comprising beam splitters, dichroic filters, and combinations thereof that can be used to analyze a single wavelength without spectrally dispersing the emission light 57. Example of such apparatus can be found in U.S. Pat. Nos. 6,015,674 and 6,563,581, as well as U.S. Patent Application Publication 2003/0160957, U.S. patent Ser. Nos. 11/086,261 and 11/096,282.

In some embodiments, emission light 57 detected by detection system 54 can be sent to a data-friendly system for analysis. In some embodiments, the data-friendly system comprises at least one computer. In some embodiments, thermocycler system 50 additionally comprises at least one microprocessor operable to control the system and/or to collect data. In some embodiments, the at least one microprocessor also comprises software and devices operable for data collection; for coordination of electronic, mechanical and optical elements of the system; and for thermocycling. In some embodiments, data analysis includes organization, manipulation and reporting of measurements and derived quantities for determining relative gene expression within the sample, between samples, and across multiple runs, and the ability for data archiving, data retrieval, database analysis and bioinformatics functionality from the data collection and data analysis.

In some embodiments, methods can be performed using commercially available equipment, or modifications thereof so as to accommodate and facilitate the use of microplate 12 of the present teachings. Examples of such commercially available equipment which may be modified can include the AB 7300 Real-Time PCR System, the AB 7500 Real-Time PCR System, the AB 7500 Fast Real-Time PCR System, the AB 7900 HT Fast Real-Time PCR System, The AB Prism® 700 Sequence Detection System, and the AB 1700 Chemiluminescent Microarray Analyzer, all of which are marketed by Applied Biosystems, Foster City, Calif., USA.

As should be appreciated from the discussion above, the present teachings can find utility in a wide variety of amplification methods, such as PCR, Real-Time Time PCR, Reverse Transcription PCR (RT-PCR), Ligation Chain Reaction (LCR), Nucleic Acid Sequence Based Amplification (NASBA), Self-Sustained Sequence Replication (3SR), strand displacement activation (SDA), Q (3replicase) system, isothermal amplification methods, and other known amplification method or combinations thereof. Additionally, the present teachings can find utility for use in a wide variety of analytical techniques, such as ELISA; DNA and RNA hybridizations; antibody titer determinations; gene expression; recombinant DNA techniques; hormone and receptor binding analysis; and other known analytical techniques. Still further, the present teachings can be used in connection with such amplification methods and analytical techniques using not only spectrometric measurements, such as absorption, fluorescence, luminescence, transmission, chemiluminescence, and phosphorescence, but also calorimetric or scintillation measurements or other known detection methods. It should also be appreciated that the present teachings can be used in connection with microcards and other principles, such as set forth in U.S. Pat. Nos. 6,126,899 and 6,124,138.

In some embodiments, the present teaching provides methods and apparatus for Reverse-Transcriptase PCR (RT-PCR), which includes the amplification of a Ribonucleic Acid (RNA) target. In some embodiments, assay 1000 can comprise a single-stranded RNA target, which comprises the sequence to be amplified (such as, for example, an mRNA), and can be incubated in the presence of a reverse-transcriptase, two primers, a DNA polymerase, and a mixture of dNTPs suitable for DNA synthesis. During this process, one of the primers anneals to the RNA target and can be extended by the action of the reverse-transcriptase, yielding an RNA/cDNA doubled-stranded hybrid. This hybrid can be then denatured and the other primer anneals to the denatured cDNA strand. Once hybridized, the primer can be extended by the action of the DNA polymerase, yielding a double-stranded cDNA, which then serves as the double-stranded target for amplification through PCR, as described herein. RT-PCR amplification reactions can be carried out with a variety of different reverse-transcriptases, and in some embodiments, a thermostable reverse-transcriptases can be used. Suitable thermostable reverse transcriptases can comprise, but are not limited to, reverse-transcriptases such as AMV reverse-transcriptase, MuLV, and Tth reverse-transcriptase.

In some embodiments, at least one polynucleotide target can be amplified using isothermal amplification methods. Such isothermal amplification method can include, for example, Strand-displacement amplification, and examples of such can be found in Walker et al., Proc. Natl. Acad. Sci. USA, 89:392 (1992); and examples of such can be found in Transcription-Mediated Amplification (TMA) and examples of such can be found in Guatelli et al., Proc. Natl. Acad. Sci. USA, 87:1874 (1990); Rolling-Circle Amplification (RCA) and examples of such can be found in Fire & Xu, Proc. Natl. Acad. Sci. USA, 92:4641 (1995); Helicase-Dependent Amplification (HDA) and examples of such can be found in Vincent et al., EMBO 5(8):795 (2004); as well as Self-Sustained Sequence Replication (3SR) and examples of such can be found in Fahy et al., PCR Method Appl. 1:25-33 (1991), as well as U.S. Pat. Nos. 5,846,717; 6,001,567; 6,692,917; 6,706,471; and 6,913,881, which are marketed as Invader® technology commercially available from Third Wave Technologies, Madison, Wis., USA.

In some embodiments, at least some of the plurality of reaction chambers 70 of microplate 12 comprises a solution operable to perform multiplex PCR. In some embodiments, multiplex methods are provided wherein assay 1000 comprises a first universal primer that binds to a complement of a first polynucleotide target, a second universal primer that binds to a complement of a second polynucleotide target, a first detection probe comprising a sequence that binds to the sequence comprised by the first polynucleotide target, and a second detection probe comprising a sequence that binds to a sequence comprised by the second polynucleotide target. First and second detection probes can comprise different labels, for example, different fluorophores such as, in non-limiting example, VIC and FAM. Sequences of the first and second detection probes can differ by as little as one nucleotide, two nucleotides, three nucleotides, four nucleotides, or greater, provided that hybridization occurs under conditions that allow each detection probe to hybridize specifically to its corresponding polynucleotide target. In some embodiments, multiplex PCR can be used for relative quantification, where one primer set and detection probe amplifies a polynucleotide target and another primer set and detection probe amplifies an endogenous reference. In some embodiments, the present teachings provide for analysis of at least four polynucleotide targets in at least one of the plurality of reaction chambers 70. Some embodiments provide for analysis of a plurality of polynucleotide targets and a reference in each of the plurality of reaction chambers 70.

In some embodiments, kits can be provided comprising materials suitable for carrying out polynucleotide amplification. In some embodiments, such a kit can comprise microplate 12 and at least a reagent such as, for example, PCR master mix, such as described above herein. In some embodiments, such kits can comprise solutions packaged for preamplification of polynucleotide targets for downstream or subsequent analysis including, for example, by multiplex PCR. In some embodiments, a kit can comprise a plurality of primer sets. In some embodiments, a kit can further comprise a set of amplification primers suitable for pre-amplifying a sample of a polynucleotide target disposed in at least some of the plurality of reaction spots 10. In some embodiments, primers comprised in each of the plurality of reaction spots 10 can, independently of one another, be the same or a different set of primers.

In some embodiments, a kit can comprise at least one primer and at least one detection probe disposed in at least some of the plurality of reaction spots 10. In some embodiments, a kit can comprise a forward primer, a reverse primer, and at least one FAM labeled MGB quenched PCR detection probe disposed in at least some of the plurality of reaction spots 10. In some embodiments, a kit can comprise at least one detection probe, and at least one primer, disposed in at least some of the plurality of reaction spots 10. In some embodiments, a kit can comprise at least one forward primer, at least one reverse primer, at least one labeled MGB quenched detection probe and at least one labeled MGB quenched detection probe used as a passive internal reference disposed in at least some of the plurality of reaction spots 10. In some embodiments, a ROX labeled detection probe can be used as a passive internal reference. Some embodiments comprise other detection probes to be used as a passive internal reference. In some embodiments, any of the above mentioned kits can also comprise reagents for preamplification. In some embodiments, any of the above mentioned kits can also comprise amplification reagents. In some embodiments, any of the above mentioned kits can also comprise a polymerase and a PCR master mix. In some embodiments, a kit can comprise a data storage medium which contains information about the contents of microplate 12.

In some embodiments, a kit comprises a container containing microplate 12 comprising assay reagents on at least some of the plurality of reaction spots 10 and a separate data storage medium that contains data about the assay reagents. The assay reagents can be adapted to perform an allelic discrimination or expression analysis reaction when mixed with at least one polynucleotide target. The other reagents can be, for example, components conventionally used for PCR and can comprise non-reactive components. In some embodiments, the container can have a machine-readable label that provides information about the contents of the container.

In some embodiments, the present teachings provide methods for amplifying at least one polynucleotide target in assay 1000 comprising a plurality of polynucleotide targets, each polynucleotide target being present at very low concentration within the assay. In some embodiments, such methods can comprise the steps of applying amplification reactants to substrate 14 comprising at least some of a plurality of reaction spots 10; forming a sealed reaction chamber 70 comprising at least some of a plurality of reaction spots 10; and subjecting substrate 14 and assay 1000 to reaction conditions for amplification of the at least one polynucleotide target. In some embodiments, reaction chamber 70 can have a volume of less than about 20 nanoliters.

In some embodiments, a method comprises performing PCR on a polynucleotide target in a complex mixture of polynucleotides. In some embodiments, a method comprises simultaneously amplifying a plurality of polynucleotide targets in a complex mixture of polynucleotides. In some embodiments, a method can be conducted on microplate 12 containing the plurality of reactions spots 10, wherein each of the plurality of reaction spots 10 comprises reagents for amplifying a single polynucleotide target. In some embodiments, each of the plurality of reactions spots 10 comprises reagents for amplifying one or more polynucleotide targets that are distinct from polynucleotide targets to be amplified in other of the plurality of reaction spots 10 on microplate 12. In some embodiments, microplate 12 comprises a plurality of reaction spots 10 comprising reagents for amplifying the same individual or group of polynucleotide targets.

In some embodiments, applying of reactants to first surface 11 of substrate 14 comprises any method by which the reagents are contacted with any of the plurality of reaction spots 10 in such a manner so as to make the reactants available for amplification reaction(s) in or on any of the plurality of the reaction spots 10. In some embodiments, the reactants are applied in a substantially uniform manner, so that each of the plurality of reaction spots 10 can be contacted with a substantially equivalent amount of reagent. In some embodiments, a substantially equivalent amount of reagent applied to at least one of the plurality of reaction spots 10 is an amount which, in combination with an associated reagent, is sufficient to effect amplification of a polynucleotide target in equivalent amounts and timing with another of the plurality of reaction spots 10 on substrate 14 (consistent with the quantity and nature of polynucleotide target to be amplified in at least one of the plurality of reaction spots 10). In some embodiments, the sample and amplification reaction reagents are mixed prior to application to first surface 11. In other embodiments, the sample and amplification reagents are applied to first surface 11 separately, either concurrently or sequentially (in either order).

In some embodiments, methods of application can comprise pouring of reactants onto first surface 11 so as to substantially cover the entire first surface 11 (including the plurality of reaction spots 10 and adjacent areas on first surface 11). In some embodiments, methods of application can comprise spotting or spraying of reactants to specific reaction spots of the plurality of reaction spots 10 (such as, for example, by use of pipettes, or automated devices, such as piezoelectric pumps, for delivering microliter quantities of materials). In some embodiments, an application step can comprise a dispersion step to effect application of the reactants (or any portion thereof) across first surface 11 of substrate 14. Such dispersion step can include use of vacuum, centrifugal force, and combinations thereof. In some embodiments, a sample can be applied by pouring the sample on substrate 14. In some embodiments, a sample can be applied by placing microplate 12 in a flow cell and circulating the sample across first surface 11 of substrate 14. In some embodiments, an amplification reagent mixture can be applied by spraying the mixture onto first surface 11, such that the mixture adheres to the plurality of reaction spots 10 and does not adhere to adjacent hydrophobic areas on substrate 14.

In some embodiments, an application step can comprise a reactant removal step, wherein excess reactant can be removed after the reactant application. In some embodiments, a reactant removal step can be affected by use of gravity, centrifugal force, vacuum, and combinations thereof. In some embodiments, the reactant removal step can be affected using a wiping device, such as a squeegee, which can be drawn across the surface of substrate 14 so as to remove excess reactant. As will be appreciated by one of skill in the art, the wiping device should be contacted to the surface with sufficient force so as to effect removal of excess reactant, without also removing all reactants and associated reagents from the plurality of reaction spots 10. In some embodiments, the application step can further comprise an incubation step, after the reactant can be applied to first surface 11 but before a reactant removal step, if needed, so as to allow a sample to hybridize with target specific reagents associated with at least one of the plurality of reaction spots 10. In some embodiments, the incubation can comprise allowing a sample to remain in contact with first surface 11 from about 0.5 to about 50 hours. In some embodiments, an application step can comprise applying a sample, incubating the sample and associated reagents in at least one of the plurality of reaction spots 10, and applying an amplification reagent mixture. In some embodiments, the incubation can further comprise heating or cooling substrate 14 to effect a reaction on or in at least one of the plurality of reaction spots 10. In some embodiments, methods can additionally comprise a reactant removal step after the incubating step and before the applying step.

In some embodiments, at least one polynucleotide target in a sample can be preamplified before the applying step, so as to increase the concentration in the sample. In some embodiments, a method can comprise methods wherein a portion of a sample can be preamplified prior to a distributing step, by (1) mixing the portion with reactants comprising a plurality of PCR primers corresponding to the PCR primers in a subset of the plurality of reaction spots 10 on substrate 14; (2) thermocycling the mixture so as to produce a pre-amplified sample; and (3) distributing the preamplified sample to the at least some of the plurality of reaction spots 10. In some embodiments, the plurality of PCR primers comprises from about 100 to about 1000 primer sets. In some embodiments, the plurality of PCR primers comprises from about 2 to about 50 primer sets.

In some embodiments, the methods of attaching a polynucleotide to hydrophobic substrate 14 discussed above can be used to construct a microarray. Microarrays of biomolecules, such as, for example, DNA, RNA, cDNA, polynucleotides, oligonucleotides, proteins, and the like, are state-of-the-art biological tools used in the investigation and evaluation of biological processes, including gene expression and mutation for analytical, diagnostic, and therapeutic purposes. In some embodiments, a microarray comprises a plurality of synthesized or deposited polynucleotides on first surface 11 of substrate 14 in an array pattern of features. In some embodiments, the support-bound polynucleotides called probes, which function to bind or hybridize with a sample of polynucleotide material can be, for example, a moiety in a mobile phase, which can be called a target in hybridization experiments. However, in some embodiments, some investigators also use the reverse definitions, referring to the surface-bound polynucleotides as targets and the solution sample of polynucleotide as probes. Further, in some embodiments, some investigators bind a target sample under test to a microarray substrate 14 and put the polynucleotide probes in solution for hybridization. In some embodiments, polynucleotide bound to at least one of a plurality of reaction spots 10 of microarray substrate 14 can be between about 10 and about 70 nucleotides, or about 20 to about 30 nucleotides. In some embodiments, a plurality of probes and/or targets in each location in an array on microarray substrate 14 can be known as a feature. In some embodiments, a feature can be a locus onto Which a large number of probes and/or targets all having the same monomer sequence can be immobilized. In some embodiments, one of the plurality of reaction spots 10 can comprise a feature. In some embodiments, first surface 11 comprising a plurality of reaction spots 10 can be contacted with one or more targets under conditions that promote specific, high-affinity binding of the target to one or more of the probes located at least one of a plurality of reaction spot 10. In some embodiments, the targets can be labeled with a detection probe, such as, for example a fluorescent tag, dye or fluorophore, so that the targets can be detectable with scanning equipment after a hybridization assay. In some embodiments, the detection probe can comprise an antibody. In some embodiments, microarray substrate 14 comprise a plurality pf reaction spots, each reaction spot comprising a first probe designed to hybridize with a first target comprising a detection probe comprising a fluorophore and a second probe designed to hybridize with a second target comprising a detection probe comprising a label or a tag that is not fluorophore. In some embodiments, the first probe comprises about half of the nucleotide length as the second probe. In some embodiments, the second target comprises a detection probe comprising an antibody. In some embodiments, the second target comprises a detection probe comprising a chemiluminescence moiety. In some embodiments, a detection probe comprises a chemiluminescence moiety. In some embodiments, a microarray can be prepared as a means to match known and unknown DNA samples based on hybridization principles, for example, to identify gene sequences or to determine gene expression levels. In some embodiments, a microarray can be made by spotting reaction spots 10 of suspended, purified polynucleotide onto first surface 11 of substrate 14. Some examples of microarray can be found in U.S. Pat. Nos. 5,143,854; 5,445,934; 5,700,642; 5,744,305; 6,203,989; 6,319,674; and 6,927,029; as well as examples of commercially available microarrays marked by Applied Biosystems, Aglilent, Xeotron, Luminex, and Affymetrix. Other examples of microarray construct protocol can be found at National Genome Research Institute (now research.nhgri.nih.gov/microarray/protocols.html) and the Institute for Genomic Research (www.tign.org/microarray/protocolsTIGR.shtml).

In some embodiments, scanning equipment used for microarray analysis, such as scanning fluorometers can comprise an excitation light source, an optical system for directing light to and from a sample being scanned, a detection system and optionally an analysis system. In some embodiments, to analyze a microarray after a hybridization assay, a scanner scans excitation light from its excitation light source across the microarray. The light excites the detection probes on the hybridized biomolecules. In some embodiments, the excited detection probes emit emission at one or more particular wavelengths. The emission light from the hybridized biomolecules can be detected and measured by a detection system and the measurements are analyzed by analysis equipment to determine the results of the hybridization assay. Example of such apparatus can be found in U.S. Pat. Nos. 6,741,344; 6,583,424; 6,407,858; 6,794,658; and 6,545,264.

In some embodiments, such a suitable apparatus comprises a platform for supporting a microarray substrate 14, a focusing element selectively alignable with at least one of the plurality of reaction spots 10 on a microarray substrate 14, an excitation source to produce an excitation beam that is focused by the focusing element into a selected reaction chamber when the focusing element is in the aligned position, and a detection system to detect a selected emitted energy from a sample placed in at least one of the plurality of reaction spots 10. In some embodiments, the focusing element can be selected in an aligned position or an unaligned position relative to at least one of the plurality of reaction spots 10. Also, some embodiments include at least one of the platform and the focusing element that rotates about a selected axis of rotation to move the focusing element between the aligned position and the unaligned position. Examples of such apparatus can be found in U.S. Pat. Nos. 4,683,195; 5,575,610; 5,602,756; and 6,563,581 and U.S. Patent Application Publication No. 2003/0160957.

### EXAMPLE 1

An exemplary amplification method of these teachings is performed using a surface-treated microscope slide, supplied by Scienion AG (Berlin, Germany), on which discrete reaction spots comprising hydrophilic areas are created. Each reaction spot is essentially circular in shape, having a diameter of about 160 μm. An array of 30,000 reaction spots is formed on the surface of the slide. Sets of PCR primers and detection probes, for hybridizing with known oligonucleotides, such as, for example, polynucleotide targets, are then deposited on the hydrophilic areas of the reaction spots and covalently linked to the reaction spots through a cleavable disulfide linker, forming reaction spots. A unique set of primers and detection probes is deposed on each reaction spot.

A sample containing a mixture of polynucleotide is then flooded across the surface of the slide, contacting the reaction spots. The sample is allowed to incubate for about twelve hours, after which excess sample is removed from the surface using a squeegee. An amplification reagent mixture comprising a disulfide cleavage agent (TaqMan® Universal Master Mix, marketed by Applied Biosystems, Foster City, Calif., USA, modified to comprise an elevated amount of dithio threitol) is then sprayed onto the surface of the slide, adhering to the reaction spots. The dithio threitol cleaves the disulfide linkage of the covalently attached polynucleotides that are primers and detection probes, thereby releasing the primers and detection probes for an amplification reaction. The volume of PCR reactants in each reaction spot is less than 2 nl. The surface is then flooded with mineral oil to seal the reaction spots and create reaction chambers and the slide placed in an instrument which is able to illuminate and scan finely-spaced reaction spots and an example of such an instrument is illustrated in FIG. 9. The reaction chambers are then thermally cycled. The number of cycles is then determined for amplicons to be produced in each reaction chamber reaching detection levels, thereby allowing qualitative and quantitative analysis of polynucleotide targets in the sample according to conventional analytical methods.

### EXAMPLE 2

A microplate is made according to these teachings by applying discrete reaction spots of agarose onto a polycarbonate plastic substrate. A solution is made comprising 3% (by weight) of agarose having a melt point ≦65° C., supplied as NuSieve GTG, by FMC BioProducts (Rocland, Me., USA). The solution is then spotted onto the surface of the substrate in an array comprising 15,000 reaction spots. The microplate is then used in a method according to Example 1. In this method, high resolution blend agarose 3:1, and monoclonal anti-biotin-agarose, supplied by Sigma (St. Louis, Mo., USA) can be substituted for the low melt agarose, with substantially similar results. In some embodiments, biotinylated polynucleotides such as primers and detection probes are used.

### EXAMPLE 3

A microplate is made according to these teachings, by cutting an optical adhesive cover comprising a plastic material, to the size of a standard glass microscope slide, and pasting the cover to the standard glass microscope slide. Heat and pressure is applied while smoothing the cover over the glass surface in order to expel air bubbles between the cover and glass surface. 2 uL droplets of 1% low melting agarose are delivered onto the plastic surface of the cover at a 4500 μm pitch in a matrix and dried at low heat on a hot plate to create a plurality of reaction spots. The plastic surface is rinsed with deionized water. A matrix of water droplets is retained on the reaction spots on the plastic surface when the excess of water was removed. 2 uL of RNase P TaqMan® reaction mix, supplied by Applied Biosystems (Foster City, Calif., USA) with human genomic DNA is then added onto each reaction spot and covered with mineral oil to seal the reaction spots and create reaction chambers. Thermocycling and fluorescence detection are then carried out using an instrument using a method as described in Example 1 or other apparatus, such as, for example, as illustrated in FIG. 9, with conditions that are compatible with microplate materials and the contents of the reaction chambers.

### EXAMPLE 4

A microplate can be made according to these teachings, by applying discrete reaction spots of PVA onto a polycarbonate plastic substrate. A solution can be made comprising 0.01% (by weight) of PVA having a melt point 258° C., supplied as Celvol by Celanese. The solution can then be spotted onto the surface of the substrate in an array comprising 15,000 reaction spots. The reaction spots comprising PVA can be treated with polymaleic anhydride and then can be coupled with polynucleotides possessing a terminal nucleotide attached to a linker with an activated amine. The final conjugation step can be made by reacting the polynucleotide-linker molecules with the reaction spots comprising PVA in the presence of EDC and Sulfo-NHS. The microplate can then used in a method according to Example 1 or any other PCR methods of these teachings.

### EXAMPLE 5

A microplate can be made according to these teachings, by spotting a solution of PVA conjugated polynucleotides onto the surface of the substrate creating an array comprising at least 10,000 reaction spots. 2 μL of RNase P TaqMan® reaction mix, supplied by Applied Biosystems (Foster City, Calif., USA) with human genomic DNA can then be added onto each reaction spot and covered with mineral oil to seal the reaction spot and create a reaction chamber. The reaction chambers can then be thermocycled using a PCR instrument according to methods of these teachings.

### EXAMPLE 6

Polystyrene-phosphoramidite is made by phosphorylating a hydroxyl terminated polystyrene (MW 10K). The polystyrene-phosphoramidite is then coupled to the solid phase bound polynucleotide via a standard solid phase polynucleotide synthesis. The resulting polystyrene-polynucleotide (ps-poly) was then cleaved with conc. NH₄OH. The mixture was then dried down to dryness. The desired ps-poly was then extracted out of the mixture of solid support and uncoupled polynucleotides with DMF. Due to the very low solubility of unconjugated polynucleotides in DMF, the DMF extract can be used directly. Two types of ps-poly were prepared. One type has single ps-poly (1 ps-poly) moiety as illustrated in FIG. 12(a) and the other type has two ps-poly (2 ps-poly) moieties attached to the polynucleotide as illustrated in FIG. 12(b).

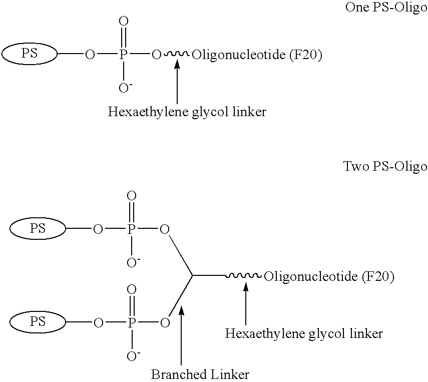

### EXAMPLE 7

An exemplary hybridization assay was carried out disclosed method. Two dye labeled probes were used. One is a complimentary 5′FAM-F20 probe (5FAM_F20p), the other non-complimentary 3′FAM-F317 probe (3FAM-F317p). Hybridization assay was carried out in 1× HBP hybridization buffer (Applied Biosystems) on a shaker at 38° C. Washing step in 1× TE buffer, pH 8 was carried out on a vortexer at room temperature. The hybridization and washing steps were done in hybridization chambers (Schleicher & Schuell) fixed onto the polyolefin covered slides.

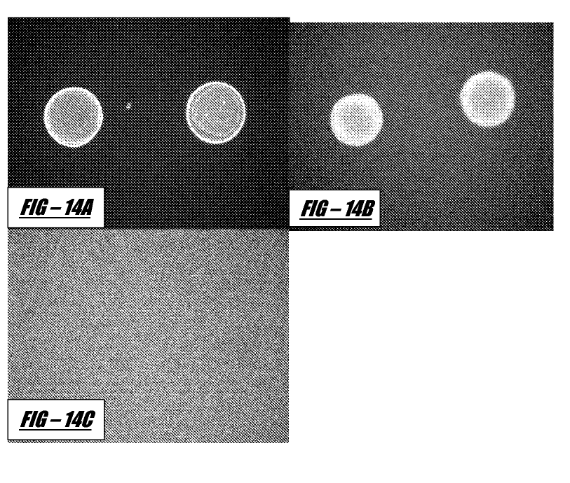

FIGS. 13(a)-(h) show microscopic images of ps and ps-poly spotted slide after hybridization in 1× HBP hybridization buffer at 38° C. for 6 hours. The spots were manually made from ps and ps-poly in DMF. 1 uM probe solutions were used for the hybridization assay. In FIG. 13, (a) and (b) are fluorescence and transmission images of ps only spot hybridized with 5FAMF20p; (c) and (d) fluorescence and transmission images of 1 ps-F20c spot hybridized with 5FAMF20p; (e) and (f) fluorescence and transmission images of 1 ps-F20c spot hybridized with 5FAMF317p; (g) and (h) fluorescence and transmission images of 2 ps-F20c spot hybridized with 5FAMF20p.

One nanoliter droplets of ps only and ps-poly solutions can also be printed on polyolefin slides for hybridization assays. Aqueous solutions of ps, 1 ps-F20c, and 2 ps-F20c were used for the printing on TopSpot instrument. The 1 nL printed slide was hybridization with 1 uM 5FAM-F20p and 3FAM-F317p in 1× HBP hybridization buffer at 38° C. for 24 hours. Fluorescence images taken on a Zeiss microscope using filters for a FAM signal of these spots after hybridization with 5FAM-F20p are shown in FIGS. 14(a)-(c), (a) 1 ps-F20c in 1 nLH2O hybridized with 5FAM-F20p; (b) 2 ps-F20c in 1 nLH2O hybridized with 5FAM-F20p; (c) 0.01 mM ps only in 1 nLH2O hybridized with 5FAM-F20p. No fluorescence signal was observed under the microscope for spots that were hybridized with non-complimentary 3FAM-F317p probe. The adsorption of ps-poly on polystyrene is strong enough to withstand thermocycling condition for PCR reactions. 1 ps-F20c spots are still visible on polyolefin surface after heated in 1× TE at 90° C. overnight.

### EXAMPLE 8

2 nL TaqMan RNase P reactions were spotted on pre-patterned Scienion slide by non-contact printing using TopSpot/E Arrayer instrument from HGS-IMIT (Freiburg, Germany). The Scienion slide contained hydrophilic reaction spots of 200 μm in diameter on an otherwise hydrophobic surface. The slide was part of a slide apparatus, illustrated in FIG. 11, which comprises microplate 12, cover 81, and a 250 μm silicone rubber sealing gasket 83. However, the volume created by the rubber gasket was fully filled with biological grade mineral oil. Two factors can affect the evaporation of water through oil layer in such cases: i.e. the solubility of water in oil and the permeability of water through the oil layer. The apparatus shown in FIG. 11 can alleviate the evaporation or partition of nanoliter aqueous droplets into oil and can enable successful PCR reactions in such small volumes on a surface.

The permeability of water through the oil overlay was not considered as the main reason for the disappearance of nanoliter aqueous droplets on the surface. This is supported by observations that 2 nL aqueous droplets on glass slide surface can survive for over 2 hours when heated at 95° C., if they are covered by a thin layer of mineral oil that is exposed to open air. However, the oil layer has to be thin such as to barely form a continuous layer on the surface.

After filling the void area with oil, excessive oil is removed by pipetting or other means, leaving only a very thin layer of oil covering the PCR reaction droplets during thermocycling. 2 nL TaqMan reaction spots were printed on the Scienion slide by non-contact printing using TopSpot instrument. The droplets were well preserved during the thermocycling and DNA target amplification. The Real-Time PCR data was collected on PCR instrument comprising a scanning laser or other thermocycler system 50 as illustrated in FIG. 9 with two PMTs for each of the FAM and ROX signals respectively.

All literature and similar materials cited in this application, including but not limited to, patents, patent applications, articles, books, treatises, and internet web pages, regardless of the format of such literature and similar materials, are expressly incorporated by reference in their entirety for any purpose. In the event that one or more of the incorporated literature and similar materials differs from or contradicts this application, including but not limited to defined terms, term usage, described techniques, or the like, this application controls.

The examples and various embodiments described herein are exemplary and not intended to be limiting in describing the full scope of compositions, apparatus, systems, and methods of these teachings. Equivalent changes, modifications and variations of any of the various embodiments, materials, compositions and methods can be made within the scope of the present teachings, with substantially similar results.

1. A method for performing PCR on a liquid biological sample comprising a polynucleotide target, the method comprising:
providing a substrate having a hydrophobic surface, said hydrophobic surface having a plurality of reaction spots, wherein a primer is anchored to at least one of said plurality of reaction spots, said primer is designed to hybridize with the polynucleotide target;
loading the liquid biological sample comprising the polynucleotide target onto said plurality of reaction spots;
loading a PCR reagent mixture onto said plurality of reaction spots;
forming a reaction chamber having a volume less than 5 nanoliters, said reaction chamber comprising said primer anchored to said at least one of said plurality of reaction spots, said PCR reagent mixture, a portion of the liquid biological sample comprising the polynucleotide target, and a sealing liquid covering said at least one of said plurality of reaction spots, said PCR mixture and said portion of the liquid biological sample;
releasing said primer from said one of said plurality of reaction spots;
hybridizing said primer to the polynucleotide target; and
amplifying the polynucleotide target.

2. The method according to claim 1, further comprising anchoring a detection probe to said at least one of the plurality of reaction spots, said detection probe designed to hybridize with the polynucleotide target, and to emit a signal indicative of amplification of the polynucleotide target.

3. The method according to claim 2, further comprising detecting said signal indicative of amplification of the polynucleotide target.

4. The method according to claim 1, further comprising spotting said hydrophobic surface with a hydrophilic material to create said plurality of reaction spots.

5. The method according to claim 4, further comprising producing at least 30,000 reaction spots.

6. The method according to claim 1, wherein said PCR reagent mixture further comprises a polymerase.

7. The method according to claim 1, wherein said loading a PCR reagent mixture onto the plurality of reaction spots, further comprises spraying said PCR reagent mixture onto said hydrophobic surface.

8. The method according to claim 1, further comprising applying a thin film of a sealing fluid over said reaction chamber resulting in a barely continuous layer of said sealing fluid on said reaction chamber and sealing said reaction chamber from other of said plurality of reaction spots.

9. The method according to claim 1, further comprising cycling a temperature of said reaction chamber from a denaturation temperature to an annealing temperature to an extension temperature.

10. The method according to claim 1, further comprising performing real time PCR on said portion of the liquid biological sample comprising the polynucleotide target.

11. The method according to claim 1, further comprising removing an excess of the liquid biological sample prior to said forming a reaction chamber.

12. The method according to claim 1, further comprising removing an excess of said PCR reagent mixture prior to said forming of a reaction chamber.

13. The method according to claim 1, wherein said reaction chamber has a volume of the liquid biological sample of about 0.1 nanoliters to about 5 nanoliters.

14. The method according to claim 1, wherein said substrate comprises a material selected from glass, plastic, silicon, quartz, nylon, metal, borosilicate, fused silica, polytetrafluoroethylene, polyethylene, polypropylene, polycarbonate, polyolefin, polyetherketone, polydimethyl siloxane, polystyrene, and combinations thereof.

15. A method for fabricating a microarray for analyzing a target in a liquid biological sample, the method comprising:
providing a substrate comprising a hydrophobic surface;
spotting onto said hydrophobic surface a hydrophilic solution comprising a polynucleotide conjugated to a functional moiety of said hydrophilic solution to produce a plurality of reaction spots on said hydrophobic surface;
controlling an amount of said hydrophilic solution that is spotted on said hydrophobic surface such that each of said plurality of reaction spots has a capacity to retain less than 5 nanoliters of an aqueous solution; and
providing a detection probe operably emitting a signal indicating hybridization of said polynucleotide to the target.

16. The method according to claim 15, wherein said polynucleotide is a probe operable for microarray hybridization analysis.

17. The method according to claim 15, wherein said polynucleotide is a primer operable for a PCR amplification.

18. The method according to claim 15, further comprising anchoring said polynucleotide to said hydrophobic surface of said substrate.

19. The method according to claim 15, further comprising cleaving said polynucleotide, making said polynucleotide available for a reaction in the liquid biological solution.

20. A microplate for use in performing PCR on a target, the microplate comprising:
a substrate comprising a hydrophobic surface;
a plurality of hydrophilic reaction spots on said hydrophobic surface of the substrate, each of said plurality of hydrophilic reaction spots having a capacity to retain less than 0.5 nanoliters of an aqueous solution;
at least one primer anchored to each of said plurality of hydrophilic reaction spots; and
a detection probe anchored to each of said plurality of hydrophilic reaction spots.

21. The microplate according to claim 20, further comprising a liquid biological sample, wherein at least a portion of the liquid biological sample is retained on at least one of said plurality of hydrophilic reaction spots.

22. The microplate according to claim 21, further comprising a sealing liquid operably covering and sealing said liquid biological sample retained on at least one of said plurality of hydrophilic reaction spots, said sealing liquid isolating said liquid biological sample retained on at least one of said plurality of hydrophilic reaction spots from other of said plurality of hydrophilic reaction spots.

23. The microplate according to claim 20, further comprising at least one amplification reagent retained on at least one of said plurality of hydrophilic reaction spots.

24. The microplate according to claim 20, further comprising a plurality of reaction chambers, each reaction chamber comprising:
one of said plurality of hydrophilic reaction spots;
said at least one primer;
said detection probe;
an amplification reagent;
a portion of a liquid biological sample; and
a sealing liquid operably covering and sealing said one of the plurality of hydrophilic reaction spots, said primer set, said detection probe, said amplification reagent, and said portion of a liquid biological sample.

25. The microplate according to claim 20, wherein said substrate comprises a material selected from glass, plastic, silicon, quartz, nylon, metal, borosilicate, fused silica, polytetrafluoroethylene, polyethylene, polypropylene, polycarbonate, polyolefin, polyetherketone, polydimethyl siloxane, polystyrene, and combinations thereof.
